(19) United States
(12) Patent Application Publication
(10) Pub. No.: US 2023/0365934 A1
(43) Pub. Date: Nov. 16, 2023

(54) CELL POPULATION PRODUCTION METHOD

(71) Applicants: JBM Incorporation, Tokyo (JP); Mebiol Inc., Kanagawa (JP); GN Incorporation Co Ltd, Yamanashi (JP)

(72) Inventors: Shojiro KATOH, Tokyo (JP); Hiroshi YOSHIOKA, Kanagawa (JP); Samuel JK ABRAHAM, Yamanashi (JP)

(21) Appl. No.: 18/027,892

(22) PCT Filed: Sep. 22, 2021
(86) PCT No.: PCT/JP2021/034677

§ 371 Date: Mar. 22, 2023

(30) Foreign Application Priority Data
Sep. 23, 2020 (JP) 2020-158938
Apr. 15, 2021 (JP) 2021-069260

(51) Int. Cl. C12N 5/077

(52) U.S. Cl. CPC C12N 5/0655; C12N 2533/30

(57) ABSTRACT

A first method is provided for increasing the tissue regeneration capacity of a cell population, a cell population production method that includes the first method, and a cell population produced using the production method. The methods for increasing the tissue regeneration capacity of a cell population can include, for example, a step for incubating a cell population in a gel-like thermoreversible polymer solution.

### CROSS-REFERENCE TO RELATED APPLICATIONS

This application claims priority to International Application No. PCT/JP2021/034677, filed Sep. 22, 2021, and claims the benefit of Japanese Application No. 2020-158938, filed Sep. 23, 2020, and Japanese Application No. 2021-069260, filed Apr. 15, 2021, each application of which is hereby incorporated herein by reference in its entirety.

### SEQUENCE LISTINGS

The instant application contains a sequence listing which has been submitted in ASCII format via EFS-Web in the parent International Application No. PCT/JP2021/034677, filed Sep. 22, 2021, which is hereby incorporated by reference in its entirety. Said ASCII copy, created on Oct. 6, 2021, is named KUZUP012US01_SEQUENCES_PCT_JPDXMLD0001-seql.app and is 15,558 bytes in size. A request for transfer of the ASCII copy to the instant application has been made under 37 CFR 1.821(e).

### BACKGROUND

### Field of the Invention

The present invention relates to a method for increasing tissue regeneration capacity of a cell population, a production method of a cell population including the method, and a cell population produced by the production method.

### Background Technology

In recent years, cell therapies for various diseases have been developed. In particular, in areas of disease where organ transplants, etc. have been traditionally carried out, the introduction of cell therapy is being actively studied in such areas due to the constant shortage of donors.

For example, in patent literature 1, a therapy of liver disease using bioprinted liver-derived cell populations is studied. In patent literature 2, a therapy of cardiac disease by cell populations using induced pluripotent stem cells is studied.

However, there have been problems for the cell populations used in these therapies that it is not possible to constantly supply cells with the desired phenotype at an applicable portion, or to repair and maintain biological tissues for a long period of time.

### PRIOR ART DOCUMENTS

### Patent Literature

[Patent literature 1]

Japanese Unexamined Patent Application Publication (Translation of PCT Application) No. 2016-514968
[Patent literature 2]

Japanese Unexamined Patent Application Publication (Translation of PCT Application) No. 2013-507936

### SUMMARY OF THE INVENTION

### Problems to be Solved by the Invention

The present invention addresses the problems of providing a method for increasing tissue regeneration capacity of a cell population, a production method of a cell population including the method, and a cell population produced using the production method.

### Means of Solving the Problems

The present inventors have conducted diligent research to solve the above problems, and found that incubating a cell population in a thermoreversible polymer solution in a gel state can increase the tissue regeneration capacity of the cell population, and completed the invention.

In other words, the present invention relates to those listed below:
[1] a method for increasing the tissue regeneration capacity of the cell population, comprising a step of incubating the cell population in a thermoreversible polymer solution in a gel state;
[2] the method according to [1], wherein the method for increasing the tissue regeneration capacity of the cell population is one or more methods selected from the group consisting of a method for removing senescent cells in the cell population, a method for maintaining the morphology of the cells constituting the cell population, a method for maintaining or increasing content of stem cells and/or somatic stem cells with high differentiating capacity in the cell population, and a method for maintaining or increasing expression of one or more genes in the biological tissue from which the cell population is derived;
[3] the method according to [2], wherein removing senescent cells is reducing β-galactosidase positive cells in the cell population and/or extending the average telomere length of the cell population;
[4] the method according to [2] or [3], wherein the morphology of the cells is expressed by one or more indicators selected from the group consisting of a shape of the cells constituting the cell population, nuclear localization, and size ratio between the cell and the nucleus;
[5] the method according to any one of [2] to [4], wherein the content of the stem cells in the cell population is a 1-2 fucose amount of the cells constituting the cell population, and the content of somatic stem cells with high differentiating capacity is α 2-6 sialic acid amount of the cells constituting the cell population;
[6] the method according to any one of [1] to [5], wherein one or more genes expressed in the biological tissue from which the cell population is derived are selected from SOX9, COL2A1, miR140, and miR21;
[7] the method according to any one of [1] to [6], wherein the cell population is a cell population derived from cartilage tissue;
[8] the method according to [1] to [7], wherein the thermoreversible polymer is obtained by binding hydrophilic blocks and a plurality of blocks having cloud point selected from a group of a copolymer of polypropylene oxide or propylene oxide with other alkylene oxide, a copolymer of poly N-substituted acrylamide derivatives, poly N-substituted methacrylamide derivatives, N-substituted acrylamide derivatives or N-substituted methacrylamide derivatives, polyvinyl methyl ether, and partially acetylated polyvinyl alcohol;
[9] a production method of cell populations, including the method of [1] to [8];
[10] a cell population produced by the method according to [9].

### Effect of the Invention

The method of the present invention enables followings:
removing senescent cells in the cell population;
maintaining the morphology of the cells constituting the cell population;
maintaining or increasing the content of stem cells and/or somatic stem cells with high differentiating capacity in the cell population;
maintaining or increasing the expression of one or more genes in the biological tissue from which the cell population is derived.

Therefore, the method of the present invention can provide high quality cell population with high replication capacity, high differentiating capacity and high expression capacity of normal genes, etc. Thus, the cell population obtained by the method of the present invention has high tissue regeneration capacity and continuous therapeutic effect for a long period of time when applied to a subject. In addition, cell population collected from different subject has different degree of cellular senescence and different content of stem cells, etc., which can be adjusted by the method of the present invention, thus therapeutic effect among subjects becomes stabilized. Furthermore, since the method of the present invention is inexpensive and simple, it can economically provide high-quality cell therapy.

### BRIEF DESCRIPTION OF THE DRAWINGS

FIGS. 1A-1C shows a dot-plot diagram of SA-β-Gal activity measured by flow cytometry in cell populations 2D cultured for 14 days (2D/Day14) (FIG. 1A) and 25 days (2D/Day25) (FIG. 1B), and in a cell population 3D cultured for 11 days with TGP gel after 2D cultured for 14 days (3D/Day25) (FIG. 1C).

FIGS. 2A-2C shows a bar graph of SA-β-Gal intensity of SA-β-Gal activity measured by flow cytometry in cell populations 2D cultured for 14 days (2D Day14) and 25 days (2D Day25), and in a cell population 3D cultured for 11 days with TGP gel after 2D cultured for 14 days (3D Day25). FIG. 2A, Total (left) shows SA-β-Gal intensity for all single cells; FIG. 2B, FSC-High (center) shows that for single cells whose FSC (forward scattered light) was above given value; and, FIG. 2C, FSC-Low (right) shows that for single cells whose FSC was below given value.

FIG. 3 shows the average telomere length of the cell population 2D cultured for 25 days (Day25-2D) and the cell population 3D cultured with TGP gel (Day25-3D).

FIGS. 4A and 4B show tissue images of samples obtained by 2D culture (FIG. 4A) and 3D culture (FIG. 4B) with TGP gel.

FIGS. 5A-5C show immunostaining images of CD44 of cell populations obtained by 2D culture and 3D culture with TGP gel. FIG. 5A shows healthy cartilage tissue, FIG. 5B shows the 2D culture, and FIG. 5C shows the 3D culture.

FIG. 6 shows changes in the amount of α2-6 sialic acid which reacts to SNA present on cell membrane of cells constituting the cell population which was 3D cultured with TGP gel.

FIG. 7 shows changes in the amount of α2-6 sialic acid which reacts to SSA present on cell membrane of cells constituting the cell population which was 3D cultured with TGP gel.

FIG. 8 shows changes in the amount of α2-6 sialic acid which reacts to TJA-1 present on cell membrane of cells constituting the cell population which was 3D cultured with TGP gel.

FIG. 9 shows changes in the α1-3 fucose amount which reacts to UEA-1 present on cell membrane of cells constituting the cell population which was 3D cultured with TGP gel.

FIGS. 10A-10F show morphologies of cell populations 2D cultured with DMEM (FIGS. 10A and 10B) and CNTPR (FIG. 10C), cell populations 3D cultured with TGP gels dissolved by DMEM (FIGS. 10D and 10E), and cell population immediately after separation from living organism (FIG. 10F). The arrows indicate nuclei.

FIGS. 11A-11D show photographs of cell populations derived from cartilage tissue, 2D cultured after 7 days and 10 days (FIGS. 11A and 11B), and cell populations derived from cartilage tissue, 3D cultured with TGP gel after 7 days and 10 days (FIGS. 11C and 11D).

FIGS. 12A and 12B show morphologies of cell populations derived from liver tissue, obtained by 3D culture with MATRIGEL (FIG. 12A) and TGP gel (FIG. 12B).

FIG. 13 shows morphologies of cell population of HELA cells obtained by 3D culture with TGP gel.

FIG. 14 is a graph showing the expression of markers p16 and p21 in 2D cultured or 3D cultured cell populations (C8R1181).

### DETAILED DESCRIPTION

### Embodiments for Carrying Out the Invention

The present invention will be described in detail below.

Unless defined otherwise herein, all technical and scientific terms used herein have the same meaning as those commonly understood by one of ordinary skill in the art.

All patents, applications, and other publications and information referenced herein are hereby incorporated by reference in their entirety. In the event of any contradiction between the publication referenced herein and the description herein, the description herein shall prevail.

The present invention relates to a method for increasing tissue regeneration capacity of a cell population and comprises a method including a step for incubating the cell population in a thermoreversible polymer solution in a gel state. “Cell population” in the present disclosure is not limited as long as it is population containing cells having growth capacity, and the cell population may be the one containing primary cells or the one containing established cells. The primary cell may be the one immediately after separation from living organism or the one grown in culture for multiple generations. The primary cell immediately after separation from living organism is preferable, from the viewpoint of containing various stem cells. Preferably, the established cell is the cell immediately after being established, from the view point of having stable trait.

In one embodiment, the cell population is not limited as long as it is cell population separated from biological tissue, and the cell population includes the one obtained from cartilage tissue, epithelial tissue, adipose tissue, thymus tissue, thyroid tissue, skeletal muscle tissue, trachea tissue, vascular tissue, lung tissue, liver tissue, gall bladder tissue, kidney tissue, ureter tissue, appendix tissue, urinary bladder tissue, urethra tissue, testicle tissue, uterus tissue, ovary tissue, digestive organ tissue (such as stomach tissue, small intestine tissue or colon tissue), heart tissue, esophagus tissue, diaphragm tissue, spleen tissue, pancreas tissue, brain tissue, spinal cord tissue, limbs periphery tissue, retina tissue, skin tissue, oral mucosa tissue, cornea tissue, limbus tissue, dental pulp tissue, vascular tissue, digestive tract tissue, greater omentum tissue, skin tissue, liver tissue, amnion, blood, lymph fluid, bone marrow fluid, etc. The one obtained from cartilage tissue is preferable from the viewpoint of being capable of enhancing tissue regeneration capacity stably.

Such cell population can include, but not limited to, somatic cells such as cartilage cells, synovial cells, osteoclasia cells, liver cells, sinusoidal endothelial cells, islet β-cells, nerves, glia, ependymal cells, retinal cells, corneal endothelial cells, heart cells, endocrine cells, white, brown or beige adipose cells, fibroblasts, muscle satellite cells, epithelial cells, endothelial cells, salivary gland cells, blood cells, lymphocyte, etc., and stem cells thereof (progenitor cells of said somatic cells, epithelial stem cells, satellite cells, intestinal stem cells, endothelial stem cells, olfactory mucosa stem cells, hair follicle stem cells, mammary gland stem cells, neural stem cells, hematopoietic stem cells, heart stem cells, tissue stem cells such as mesenchymal stem cells, etc., embryonic stem cells, oocytes, blastomeres, inner cell mass cells, embryonic germ cells, embryoid body cells, morula derived cells, teratoma (teratocarcinoma) cells, and pluripotent cells such as embryonic stem cells derived from late stage of embryogenesis with pluripotency which is partially differentiated and iPS cells, etc.). It is preferable that cartilage cells, cartilage progenitor cells and/or mesenchymal stem cells are included from the viewpoint of being capable of enhancing tissue regeneration capacity stably.

In one embodiment, the cell population may include, not limited as long as they are known established cells, for example, epithelial cells (Vero cells, MDCK cells, CHO cells, HEK293 cells, COS cells, HmLu cells and the like), tumor cells (Hela cells, VACO cells and the like), endothelial cells (HUVEC cells, DBAE cells and the like), leukocyte (HIT-T15 cells and the like), fibroblast (WI38 cells, BHK21 cells, SFME cells and the like), muscle cells (HL1 cells, C2C12 cells and the like), neuro/endocrine gland cells (ROC-1 cells, IMR-32 cells and the like) and the like. The cell population includes, for example, a cell population that has been genetically modified from the above-mentioned cells.

The cell population can be derived from any organism. Such organisms include, but are not limited to, for example, humans, non-human primates, dogs, cats, pigs, horses, goats, sheep, rodents (e.g., mice, rats, hamsters, guinea pigs, etc.), rabbits and the like.

The cell population obtained by the method of the present invention has higher tissue regeneration capacity than the one cultured by the method not including a step for incubating the cell population in a thermoreversible polymer solution in a gel state. Tissue regeneration capacity refers to the regenerative capacity in the tissue to which the cell population is applied. Tissue regeneration capacity can be quantified, after a predetermined period of time following application of the cell population to the target tissue, by observing state of the tissue at the application site, for example tissue microstructure, tissue size, size ratio between damaged tissue and normal tissue, tissue function, etc., then converting into numerals, etc.

The cell population obtained by the method of the present invention has tissue regeneration capacity of, but not limited to, 101% or higher, 105% or higher, 110% or higher, 130% or higher, 140% or higher, 150% or higher, 160% or higher, 170% or higher, 180% or higher, 190% or higher or 200% or higher compared to a cell population incubated by the method not including a step for incubating a cell population in a thermoreversible polymer solution in a gel state (referred to herein as “control cell population”).

In one embodiment, the method for increasing the tissue regeneration capacity of a cell population may be one or more methods selected from the group consisting of a method for removing senescent cells in the cell population, a method for maintaining the morphology of the cells constituting the cell population, a method for maintaining or increasing the content of stem cells and/or somatic stem cells with high differentiating capacity in the cell population, and a method for maintaining or increasing the expression of one or more genes in the biological tissue from which the cell population is derived.

In one embodiment, the method of the present invention may be a method for removing senescent cells in the cell population. In the present disclosure, “removing senescent cells” means decrease of expression amount of senescent marker or senescent marker positive cells in the cell population.

Known senescent marker can be used, for example, β-galactosidase (SA-β-Gal), p16, p21, p53, γ-H2Ax, 53BP1, etc. can be listed.

For example, while expression amount of SA-β-Gal increases according to senescence of cells, when a cell population is incubated by the method of the present invention, expression amount of SA-β-Gal or the number of SA-β-Gal positive cells of the cell population decreases compared to the control cell population.

In the cell population obtained by the method of the present invention, expression amount of senescent marker or senescent marker positive cells in the cell population decreases by, but not limited to, 99% or less, 95% or less, 90% or less, 70% or less, 60% or less, 50% or less, 30% or less, 20% or less, 10% or less, 5% or less or 1% or less, 0.5% or less, 0.2% or less compared to the control cell population.

In addition, removing senescent cells removes cells with short telomere length in the cell population, therefore the average telomere length of the cell population can be extended. Thus, in one embodiment, in the cell population from which senescent cells are removed by the method of the present invention, the average telomere length is, but not limited to, 101% or more, 102% or more, 105% or more, 110% or more, 130% or more, 150% or more, 170% or more, 190% or more, and 200% or more compared to the control cell population. Thus, it is possible to restrain phenomenon caused by senescence of cells, especially shortening of telomere length, such as stoppage of irreversible replication and/or differentiation of cells constituting the cell population.

When such cell population from which senescent cells have been removed is applied to a subject, biological tissue can be repaired for a long period of time because healthy cells are constantly supplied from applicable portion. Furthermore, since the average telomere length of cells constituting such repaired biological tissue is long, the biological tissue can be maintained for a long period of time. Thus, the cell population obtained by the method of the present invention has high tissue regeneration capacity. In addition, continuing incubation of the cell population by the method of the present invention allows the cell population to grow while restraining development of senescent cells after removal thereof. Thus, it is possible to produce cell populations which function normally for a long period of time.

In one embodiment, the method of the present invention may be a method for maintaining the morphology of cells constituting the cell population. “Maintaining the morphology of cells constituting the cell population” means that one or more parameters (indicators) relating to cell morphology selected from shape, nuclear localization, and size ratio between cell and nuclear of the cells constituting the cell population are similar to those of cells in normal biological tissue from which the cell population is derived. While, in the art, shape of the cells is classified as spherical, polygonal, elongated, etc., it is possible to consider that shape of a cell is similar when the shape thereof is classified as same as that of a cell in normal biological tissue from which the cell population is derived, for example, by visual inspection using a microscope or using known imaging software, etc.

Regarding nuclear localization, measure the location of nuclei of a cell in normal biological tissue from which the cell population is derived and that of a cell constituting the cell population to find whether they are located near the peripheral or center of cytoplasm, then it is possible to consider that nuclear localization is similar when the nucleus is located in the same manner as the cell in the normal biological tissue. Size ratio between cell and nucleus (also referred to as “C:N ratio” in the present specification) is indicated as, for example, diameter ratio, area ratio or volume ratio between cell and nucleus, it is possible to consider that size ratio between cell and nucleus is similar when mean size ratio between cell and nucleus of a cell constituting a cell population is, but is not limited to, 30% or more, 40% or more, 60% or more, 80% or more, 90% or more, and 95% similar compared to the cell in normal biological tissue from which the cell population is derived. Each parameter of aforementioned cell morphology can be measured using usual methods in the art.

Since these cells whose morphologies are maintained can normally carry out signal control and expression, when such cell population is applied to the subject, cells expressing the desired phenotype at applicable portion can be constantly supplied and it is possible to repair and maintain normal biological tissue for a long period of time.

In one embodiment, the method of the present invention may be a method for maintaining or increasing the content of stem cells and/or somatic stem cells with high differentiating capacity in the cell population. Maintaining or increasing of stem cell content may be maintaining or increasing in the amount or proportion of stem cells per unit volume in the cell population. In the present invention, “stem cell” is not limited as long as it is a cell having differentiating capacity, and includes any progenitor cell, somatic stem cell such as mesenchymal stem cell, or pluripotent stem cell such as ES cell, iPS cell, and ntES cell. The cell population obtained by the method of the present invention, in one embodiment, has a higher content of stem cells and/or a lower content of terminally differentiated cells in the cell population compared to the control cell population. Thus, in one embodiment, the method of the present invention is a method for selecting or concentrating stem cells in the cell population, and in another embodiment, a method for diversifying the degree of differentiation of the cell population.

“Differentiating capacity” refers to the capacity of a cell to change into another type of cell, such as a given progenitor cell or somatic cell, etc., when placed in an appropriate differentiation-induction state. In general, the long-term culture of somatic stem cell tends to decrease differentiating capacity along with growth capacity. Somatic stem cell with decreased differentiating capacity has lower tissue regeneration capacity because it cannot change into another type of cell such as progenitor cell or somatic cell while it has somatic stem cell marker on its cell surface.

In one embodiment, the method of the present invention can maintain or increase the content of somatic stem cells with high differentiating capacity. Maintaining or increasing of content of somatic stem cells with high differentiating capacity may be maintaining or increasing in the amount or proportion of somatic stem cells with high differentiating capacity per unit volume in the cell population. Somatic stem cells are not limited as long as they are somatic stem cells which can differentiate into terminal differentiated cells, and may be any progenitor cells such as cartilage progenitor cells, muscle satellite cells, corneal progenitor cells, or mesenchymal stem cells, etc.

Content of stem cells or somatic stem cells with high differentiating capacity can be measured by known methods, and can be quantified by measuring, not limited to, the amount of α1-2 fucose which reacts with lectins such as UEA-1 and the like, or α2-6 sialic acid which reacts with lectins such as SNA, SSA, TJA-I and the like, both of which are present in cell membrane of cell constituting the cell population, or measuring other cell surface markers which correlate with changes in the amount of said α1-2 fucose or said α2-6 sialic acid, or gene expression and the like. For example, a higher level of said α1-2 fucose present in cell membranes of the cells in the cell population means that there are more pluripotent stem cells, and a higher level of said α2-6 sialic acid means that there are more somatic stem cells with high differentiating capacity (refer to: Wang et al, Cell Res. 2011 November; 21(11):1551-63, Tateno et al, Glycobiology. 2016 December; 26(12):1328-1337, WO2016006712A1).

The cell population obtained by the method of the present invention contain more stem cells and/or somatic stem cells with high differentiating capacity than the control cell population. In one embodiment, including more stem cells or somatic stem cells with high differentiating capacity means more content of cells having α1-2 fucose or α2-6 sialic acid, or higher expression of cell surface markers which correlate with them or genes by, not limited to, 101% or more, 105% or more, 110% or more, 130% or more, 140% or more, 150% or more, 160% or more, 170% or more, 180% or more, 190% or more or 200 or more compared to the control cell population.

When such a cell population including more stem cells and/or somatic stem cells with high differentiating capacity is applied to the subject, it can repair to normal biological tissue and be maintained for a long period of time because cells differentiated from cells with high differentiating capacity can be constantly supplied at applicable portion.

In addition, when the stem cells or the somatic stem cells with high differentiating capacity is mesenchymal stem cells, therapeutic effect can be maintained for a long period of time because of their higher capacity to normalize damaged biological tissue and to recover from damage, etc., such as for anti-inflammation and for anti-fibrosis. Thus, the cell population obtained by the method of the present invention has high tissue regeneration capacity.

In one embodiment, using the method of the present invention, it is possible to maintain or increase, in cell population, expression capacity of one or more genes expressing in the biological tissue from which the cell population is derived. In other words, using the method of the present invention, it is possible to maintain or increase, in cell population, expression of genes identical to one or more genes expressing in the biological tissue from which the cell population is derived. Preferably, biological tissue from which cell population is derived is normal biological tissue.

In one embodiment, if biological tissue from which cell population is derived is cartilage tissue, such genes may be selected from, but not limited to, SOX9, COL2A1, miR140, miR21.

In the present disclosure, SOX9 (also referred to as CMD1, CMPD1, SRA1, SRXX2, and SRXY10) is a gene encoding SRY-box transcription factor 9, and the gene sequence of human SOX9 is registered as accession number: NM_000346, etc. and the sequence is indicated as SEQ ID NO: 1.

In the present disclosure, COL2A1 (also referred to as ANFH, AOM, COL11A3, SEDC, and STL1) is a gene encoding collagen type II alpha 1 chain, and the gene sequence of human COL2A1 is registered as accession number: NM_001844.5, etc. and the sequence is indicated as SEQ ID NO: 2.

In the present disclosure, mir-140 is registered as miRbase ID: Stem-loop sequence hsa-mir-140, miRbase accession number: MI0000456, and its sequence is indicated as SEQ ID NO: 3.

In the present disclosure, miR140-3p is registered as miRbase ID: Mature sequence hsa-miR-140-3p, miRbase accession number: MIMAT0004597, and its sequence is RNA sequence “uaccacaggguagaaccacgg” and indicated as SEQ ID NO: 4.

In the present disclosure, miR140-5p is registered as miRbase ID: Mature sequence hsa-miR-140-5p, miRbase accession number: MIMAT0000431, and its sequence is RNA sequence “cagugguuuuacccuaugguag” and indicated as SEQ ID NO: 5.

In the present disclosure, mir-21 is registered as miRbase ID: Stem-loop sequence hsa-mir-21, miRbase accession number: MI0000077, and its sequence is indicated as SEQ ID NO: 6.

In the present disclosure, miR21-3p is registered as miRbase ID: Mature sequence hsa-miR-21-3p, miRbase accession number: MIMAT0004494, and its sequence is RNA sequence “caacaccagucgaugggcugu” and indicated as SEQ ID NO: 7.

In the present disclosure, miR21-5p is registered as miRbase ID: Mature sequence hsa-miR-21-5p, miRbase accession number: MIMAT0000076, and its sequence is RNA sequence “uagcuuaucagacugauguuga” and indicated as SEQ ID NO: 8.

The cell population obtained by the method of the present invention has higher level of expression of one or more genes expressed in biological tissue than the control cell population. Here, high expression level of genes means that expression level of given genes is, but not limited to, 101% or more, 105% or more, 110% or more, 130% or more, 140% or more, 150% or more, 160% or more, 170% or more, 180% or more, 190% or more or 200% or more compared to the control cell population. Usual methods in the art can be used as method of measurement of gene expression levels.

When cell population which maintains or increases the expression of one or more genes expressed in such biological tissue is applied to the subject, normal biological tissue can be repaired and maintained for a long period of time because normally functioning cells can be constantly supplied at applicable portion. Thus, the cell population obtained by the method of the present invention has high tissue regeneration capacity.

For example, high expression of miR140 in cartilage tissue results in increase of miR140-derived miR140-3p or 5p in the cartilage tissue, as a result of that, denatured state of cartilage tissue is normalized through decreased expression or secretion of ECM degrading enzymes such as MMP-13 and ADAMTS-5, decreased expression or secretion of inflammatory mediators such as IL1B, IL6, and IL8, and enhanced expression of proteins, etc. involved in ECM synthesis such as SOX9, ACAN, and chondroitin sulfate N-acetylgalactosaminyltransferase 1 (CSGALNACT1). Thus, in one embodiment, when cell population derived from cartilage tissue is applied to cartilage tissue of a subject having osteoarthritis, not only normal cartilage tissue is repaired at applicable portion, but therapeutic effect can be maintained for a long period of time because the denatured state of cartilage tissue around the applicable portion can be normalized.

In addition, high expression of miR21 in cartilage tissue results in an increase of miR21-derived mi21-5p in the cartilage tissue, as a result of that, denatured state of cartilage tissue is normalized through, but not limited to, decreased expression or secretion of ECM degrading enzymes such as MMP-13 and ADAMTS-5, and enhanced expression of proteins, etc. involved in ECM synthesis such as COL2A1. Thus, as with miR140, the therapeutic effect can be maintained for a long period of time. (Refer to: Karlsen et al. Mol Ther Nucleic Acids. 2016 Oct. 11; 5(10), Miyaki et al., Arthritis Rheum. 2009 September; 60(9):2723-30, Si et al., Osteoarthritis Cartilage. 2017 October; 25(10):1698-1707, Miyaki et al., Genes Dev. 2010 Jun. 1; 24(11):1173-85, Hai et al,. J Orthop Surg Res. 2019; 14: 118, etc.)

In one embodiment, the method of the present invention can impart tissue formation capacity to cell population. “Tissue formation capacity” refers to a capacity to form, in vitro, the biological tissue from which the cell population is derived. For example, when the biological tissue from which cell population is derived is cartilage tissue, it is the capacity to form tissue, in vitro, which has the same tissue structure as healthy cartilage tissue which has cell gaps of the cell population filled with ECM.

In one embodiment, the method of the present invention can increase microRNA (miRNA) retention capacity of the cell population. MicroRNA retention capacity is a capacity that the cell population retain miRNA in the cell population without secreting them during culture. By miRNA being retained in the cell population at the time of culture, a cell population which can secrete miRNA with high concentration at and around applicable portion can be obtained when the cell population is applied to the subject.

In the present disclosure, “microRNA (miRNA)” intends to RNA of 10-25 bases which is transcribed as RNA precursor with hairpin-like structure, cleaved by dsRNA cleavage enzyme having RNase III cleavage activity, incorporated into a protein complex called RISC, and involved in mRNA translation inhibition. In addition, “miRNA” includes “miRNA” and precursor of “miRNA” (pre-miRNA, pri-miRNA), and also includes miRNA which is equivalent in biological function to the miRNA encoded thereby (for example, “miRNA” encoding homologue (i.e., homolog), mutant such as gene polymorphism, and the derivatives). Such “miRNA” encoding precursor, homologue, mutant, or derivative includes “miRNA” which can be identified by miRBase and has base sequence which hybridizes with the complementary sequence of specific base sequence under stringent condition.

miRNA includes, when cell population is derived from cartilage tissue, but not limited to, miR140, miR21, miR-125b, Has-miR-15a, miR-30a, miR-199a, miR-210, miR-221-3p, miR-92a-3p, miR-142-3p, miR-27a, miR-27b, miR26a-5p, miR-26a, miR-26b, miR-373, miR-127-5p, miR-320, miR-9, miR-634, miR-221-3p, miR-370, miR-145, miR-130A, miR-145, miR-562-5p (refer to: Zhang. et al. J Arthritis. 2017 April; 6(2). pii: 239. doi: 10.4172/2167-7921.1000239), preferably it is miR140 (including miR140-3p and -5p), miR21 (including miR21-3p and -5p), particularly preferably miR140 (including miR140-3p and -5p).

High retention capacity of miRNA means that miRNA is present at concentration of, but not limited to, 101% or more, 105% or more, 110% or more, 130% or more, 140% or more, 150% or more, 160% or more, 170% or more, 180% or more, 190% or more or 200% or more in the cell population at cultivation compared to the control cell population.

The thermoreversible polymer in the method of the present invention (also referred to as “TGP” in the present specification) refers to a polymer which forms a crosslinked structure or a network structure in a thermally reversible manner in water, and based on said structure, which has a property of being able to thermally reversibly form a hydrogel that holds in its inside a separation liquid such as water.

In addition, the hydrogel refers to a gel comprising a crosslinked or network structure made of polymers, and water that is supported or retained in said structure (herein, TGP solution in gel state is also referred to as “TGP gel”). In the present invention, since the cross-linked structure or network structure which is peculiar to thermoreversible polymers creates an environment similar to that of living organisms in water, any polymers, as long as they are thermoreversible polymers, can be used in the method of the present invention.

Sol-Gel Transition Temperature

Definition and measurement of “sol state”, “gel state” and “sol-gel transition temperature” in the present invention are based on the definitions and measurements described in the literature (H. Yoshioka et al., Journal of Macromolecular Science, A31(1), 113 (1994)). That is, the dynamic elastic modulus of a sample at an observation frequency of 1 Hz is measured by gradually changing the temperature (1° C./1 minute) from low temperature side to high temperature side, and the temperature at the point where the storage elastic modulus of the sample (G′, elastic term) exceeds the loss elastic modulus (G″, viscosity term) is defined as the sol-gel transition temperature. Generally, a state with G″>G′ is defined as a sol and a state with G″<G′ is defined as a gel. In measuring the sol-gel transition temperature, the following measurement conditions can be preferably used.

Measurement Conditions for Dynamic/Loss Elastic Modulus

Measuring device (trade name): Stress control type rheometer AR500, manufactured by TA Instruments

Concentration of sample solution (or separation liquid) (as the concentration of “hydrogel-forming polymer having sol-gel transition temperature”): 10 (weight) %

Amount of sample solution: Approximately 0.8 g

Shape/dimensions of measurement cell: Acrylic parallel disk (diameter 4.0 cm), gap 600 μm

Measurement frequency: 1 Hz

Applied stress: Within linear region.

In the present invention, the above sol-gel transition temperature is preferably higher than 0° C. and 37° C. or lower, and furthermore preferably it is higher than 5° C. and 35° C. or lower (in particular 10° C. or higher and 33° C. or lower). A TGP having such a suitable sol-gel transition temperature in water can be easily selected from specific compounds as described later according to the above-mentioned screening method (sol-gel transition temperature measurement method).

The TGP of the present invention is not particularly limited as long as it exhibits a thermoreversible sol-gel transition as described above (that is, has a sol-gel transition temperature). As specific examples of the polymer wherein its aqueous solution has a sol-gel transition temperature and reversibly exhibits a sol state at a temperature lower than the transition temperature, the following are known: for example, polyalkylene oxide block copolymers typified by a block copolymer of polypropylene oxide and polyethylene oxide, etc.; etherified cellulose such as methyl cellulose and hydroxypropyl cellulose, etc.; chitosan derivatives (K. R. Holme, et al. Macromolecules, 24, 3828 (1991)) and the like.

Suitable Hydrogel-Forming Polymer

The hydrogel-forming polymer utilizing a hydrophobic bond for crosslink formation, which can be suitably used as a TGP of the present invention, is preferably composed by binding a plurality of blocks having a cloud point and a hydrophilic block, from the viewpoint of stably maintaining the surroundings of the cell population without separating the TGP from the medium.

The hydrophilic block is preferably present in order for the hydrogel to become water soluble at a temperature lower than the sol-gel transition temperature, and the plurality of blocks having a cloud point are preferably present in order for the hydrogel to change to a gel state at a temperature higher than the sol-gel transition temperature.

In other words, a block with a cloud point dissolves in water at a temperature lower than the cloud point and becomes insoluble in water at a temperature higher than the cloud point, so that the block serves as a cross-linking point consisting of hydrophobic bonds for the formation of a gel at the temperature higher than the cloud point. That is, the cloud point derived from the hydrophobic bond corresponds to the sol-gel transition temperature of the hydrogel.

However, the cloud point and the sol-gel transition temperature do not necessarily have to match. This is because the cloud point of the above-mentioned “block having a cloud point” is generally affected by the binding between said block and a hydrophilic block.

The hydrogel used in the present invention utilizes the property that the hydrophobic bond not only becomes stronger with increasing temperature but also the change is reversible with respect to temperature. It is preferable for the TGP to have a plurality of “blocks having a cloud point”, from the viewpoint that multiple cross-linking points are formed in one molecule and a gel with excellent stability is formed, and that a cell population can grow stably against reversible temperature changes.

In contrast, as described above, the hydrophilic block in the TGP has a function of changing the TGP to be water-soluble at a temperature lower than the sol-gel transition temperature, and has a function of forming a state of a hydrogel, while preventing the hydrogel from coagulating and settling due to excessive increase in a hydrophobic binding force at a temperature higher than the transition temperature.

Furthermore, it is desirable that the TGP used in the present invention is those that can be degraded and absorbed in vivo, from the viewpoint of using the cell population of the present invention in cell therapy. That is, it is preferable that the TGP of the present invention is degraded in a living organisms by a hydrolysis reaction or an enzymatic reaction to become a low molecular weight substance harmless to the living organisms, and is absorbed and excreted.

When the TGP of the present invention is the one that is composed by binding a plurality of blocks having a cloud point and a hydrophilic block, it is preferable that at least either one of the block having a cloud point and the hydrophilic block, preferably both of them, is/are degraded and absorbed in vivo.

A Plurality of Blocks Having a Cloud Point

The block having a cloud point is preferably a block of a polymer having a negative solubility-temperature coefficient in water, and more specifically, a polymer selected from the group consisting of polypropylene oxide, copolymers of propylene oxide and other alkylene oxides, poly N-substituted acrylamide derivatives, poly N-substituted methacrylamide derivatives, copolymers of N-substituted acrylamide derivatives and N-substituted methacrylamide derivatives, polyvinyl methyl ether, and partially acetylated polyvinyl alcohol can be preferably used. From the viewpoint of stably culturing the cell population of the present invention, a poly N-substituted acrylamide derivative is preferable.

In order to make the block having a cloud point those that can be degraded and absorbed in a living organisms, it is effective to make it a polypeptide consisting of a hydrophobic amino acid and a hydrophilic amino acid. Alternatively, a polyester-type biodegradable polymer such as polylactic acid or polyglycolic acid may be used as a block having a cloud point that is degraded and absorbed in a living organisms.

The fact that the cloud point of the above polymer (block having a cloud point) is higher than 4° C. and 40° C. or less is preferable from the viewpoint of making the sol-gel transition temperature of the polymer (a compound in which a plurality of blocks having a cloud point and a hydrophilic block are bound) used in the present invention higher than 0° C. and 37° C. or lower.

Here, the cloud point may be measured as follows: for example, an aqueous solution of about 1 mass % of the above polymer (block having a cloud point) is cooled to obtain a transparent uniform solution, and the temperature is gradually increased (heating rate of about 1° C./min); then the temperature at which the solution becomes cloudy for the first time is set as the cloud point.

Specific examples of poly N-substituted acrylamide derivatives and poly N-substituted methacrylamide derivatives that can be used in the present invention are listed below. Poly-N-acroylpiperidine; poly-N-n-propylmethacrylamide; poly-N-isopropylacrylamide; poly-N, N-diethylacrylamide; poly-N-isopropylmethacrylamide; poly-N-cyclopropylacrylamide; poly-N-acryloyl-pyrrolidine; poly-N,N-ethylmethylacrylamide; poly-N-cyclopropylmethacrylamide; poly-N-ethylacrylamide.

From the viewpoint of stably incubating the cell population, poly-N-isopropylacrylamide is preferable.

The above polymer may be a homopolymer, or a copolymer of a monomer constituting the above polymer and other monomer. As other monomer constituting such a copolymer, either a hydrophilic monomer or a hydrophobic monomer can be used. In general, copolymerization with a hydrophilic monomer raises the cloud point of the product, and copolymerization with a hydrophobic monomer lowers the cloud point of the product. Therefore, by selecting these monomers to be copolymerized, it is possible to obtain a polymer having a desired cloud point (for example, a cloud point higher than 4° C. and 40° C. or lower).

Hydrophilic Monomer

Examples of the above hydrophilic monomer include N-vinylpyrrolidone, vinylpyridine, acrylamide, methacrylamide, N-methylacrylamide, hydroxyethyl methacrylate, hydroxyethyl acrylate, hydroxymethyl methacrylate, hydroxymethyl acrylate, acrylic acids and methacrylic acids having an acidic group and salts thereof, vinyl sulfonic acid, styrene sulfonic acid, etc., as well as N,N-dimethylaminoethyl methacrylate, N,N-diethylaminoethyl methacrylate, and N,N-dimethylaminopropyl acrylamide having a basic group and salts thereof, but are not limited thereto.

Hydrophobic Monomer

Meanwhile, examples of the above hydrophobic monomer include acrylate derivatives and methacrylate derivatives such as ethyl acrylate, methyl methacrylate and glycidyl methacrylate, etc., N-substituted alkyl methacrylamide derivatives such as N-n-butyl methacrylamide, etc., vinyl chloride, acrylonitrile, styrene, vinyl acetate, etc., but are not limited thereto.

Hydrophilic Block

Meanwhile, specific examples of the hydrophilic block to be bound to the above-mentioned block having a cloud point include methylcellulose, dextran, polyethylene oxide, polyvinyl alcohol, poly N-vinylpyrrolidone, polyvinylpyridine, polyacrylamide, polymethacrylamide, poly N-methylacrylamide, polyhydroxymethyl acrylate, polyacrylic acid, polymethacrylic acid, polyvinylsulfonic acid, polystyrene sulfonic acid and salts thereof; poly N,N-dimethylaminoethyl methacrylate, poly N,N-diethylaminoethyl methacrylate, poly N,N-dimethylaminopropyl acrylamide, and salts thereof, etc.

In one embodiment of the present invention, when poly-N-isopropylacrylamide is used in the thermoreversible gel as the plurality of blocks having a cloud point, an environment similar to the in vivo environment, whereby differentiating capacity and replication capacity of the cell population are increased; therefore, as the hydrophilic block that binds to the acrylamide, polyethylene oxide is preferred.

In addition, it is desirable that the hydrophilic block is degraded, metabolized and excreted in the living organisms; and hydrophilic biopolymers such as proteins, e.g., albumin or gelatin, and polysaccharides, e.g., hyaluronic acid, heparin, chitin or chitosan are preferably used.

The method for binding the block having a cloud point and the above hydrophilic block is not particularly limited; for example, this can be done by introducing a polymerizable functional group (for example, an acryloyl group) into either of the above blocks, and copolymerizing with a monomer giving the other block. In addition, a bound substance of the block having a cloud point and the above-mentioned hydrophilic block can also be obtained by block copolymerization of a monomer giving a block having a cloud point and a monomer giving a hydrophilic block. Furthermore, binding between the block having a cloud point and the hydrophilic block can be achieved by introducing a reactive functional group (for example, a hydroxyl group, an amino group, a carboxyl group, an isocyanate group, etc.) into the both blocks in advance, and binding the both blocks by a chemical reaction.

At this time, a plurality of reactive functional groups are usually introduced into the hydrophilic block. In addition, binding between polypropylene oxide having a cloud point and a hydrophilic block is achieved by, for example, repeatedly and sequentially polymerizing the propylene oxide and the monomer constituting the “other hydrophilic block” (for example, ethylene oxide) by anionic polymerization or cationic polymerization, to obtain a block copolymer in which polypropylene oxide and the “hydrophilic block” (for example, polyethylene oxide) are bound.

Such a block copolymer can also be obtained by introducing a polymerizable group (for example, an acryloyl group) at the terminal of polypropylene oxide and then copolymerizing with a monomer constituting the hydrophilic block.

Furthermore, the polymer used in the present invention can also be obtained by introducing a functional group capable of binding reaction with a functional group (for example, a hydroxyl group) at the terminal of polypropylene oxide into a hydrophilic block and reacting both of them. In addition, the TGP used in the present invention can also be obtained by connecting a material such as Pluronic (registered trademark) F-127 (trade name, manufactured by Asahi Denka Kogyo Co., Ltd.) in which polyethylene glycol is bound to both terminals of polypropylene glycol.

The polymer of the present invention in the embodiment comprising the blocks having a cloud point is completely dissolved in water and shows a sol state at a temperature lower than the cloud point, because the above-mentioned “blocks having a cloud point” together with the hydrophilic block existing in the molecule are water-soluble at that temperature. However, when the temperature of the aqueous solution of this polymer is heated to a temperature higher than the above-mentioned cloud point, the “blocks having a cloud point” existing in the molecule become hydrophobic, and they are associated between different molecules due to hydrophobic interaction.

On the other hand, since the hydrophilic block is water-soluble even at this time (when heated to a temperature higher than the cloud point), the polymer of the present invention produces a hydrogel having a three-dimensional network structure in water with a site of hydrophobic association between the blocks having a cloud point as the cross-linking point. When the temperature of this hydrogel is cooled again to a temperature lower than the cloud point of the “blocks having a cloud point” existing in the molecule, the blocks having the cloud point become water-soluble, the cross-linking points due to the hydrophobic association are released, and the hydrogel structure disappears; and the TGP of the present invention becomes a complete aqueous solution again.

Thus, the sol-gel transition of the polymer of the present invention in a preferred embodiment is based on reversible changes between hydrophilicity and hydrophobicity at the cloud point of the blocks having a cloud point existing in the molecule; therefore, the polymer of the present invention has complete reversibility in response to temperature changes. According to studies by the present inventors, the above-mentioned delicate hydrophilic-hydrophobic balance of the TGP in water is considered to contribute to the stability of cells when they are stored at low temperature.

Solubility of Gel

As described above, the hydrogel-forming polymer of the present invention comprising at least a polymer having a sol-gel transition temperature in an aqueous solution shows substantial water insolubility at a temperature (d ° C.) higher than the sol-gel transition temperature, and reversibly shows water solubility at a temperature (e ° C.) lower than the sol-gel transition temperature.

The above-mentioned high temperature (d ° C.) is preferably 1° C. or higher than the sol-gel transition temperature, and more preferably 2° C. or higher (in particular 5° C. or higher). In addition, the above-mentioned “substantial water insolubility” means that the amount of the above-mentioned polymer dissolved in 100 ml of water at the above-mentioned temperature (d ° C.) is preferably 5.0 g or less (furthermore, 0.5 g or less, and in particular 0.1 g or less).

Meanwhile, the above-mentioned low temperature (e ° C.) is preferably 1° C. or more (absolute value) lower than the sol-gel transition temperature, and furthermore preferably 2° C. or lower (in particular 5° C. or lower). In addition, the above-mentioned “water solubility” means that the amount of the above-mentioned polymer dissolved in 100 ml of water at the above-mentioned temperature (e ° C.) is preferably 0.5 g or more (furthermore, 1.0 g or more). Furthermore, “reversibly shows water solubility” means that even after the above-mentioned TGP aqueous solution is once gelled (at a temperature higher than the sol-gel transition temperature), it shows the above-mentioned water solubility at a temperature lower than the sol-gel transition temperature.

It is preferable that the 10% aqueous solution of the above polymer exhibits a viscosity of 10 to 3,000 centipoise (furthermore, 50 to 1,000 centipoise) at 5° C. Such viscosity is preferably measured under the following measurement conditions, for example.

Viscometer: Stress control type rheometer (model name: AR500, manufactured by TA Instruments)

Rotor diameter: 60 mm

Rotor shape: Parallel flat plate

Even if the aqueous solution of the TGP of the present invention is gelled at a temperature higher than the above-mentioned sol-gel transition temperature and then immersed in a large amount of water, the gel does not substantially dissolve. The above-mentioned characteristics of the hydrogel formed by the above-mentioned TGP can be confirmed, for example, as follows.

That is, 0.15 g of the TGP is dissolved in 1.35 g of distilled water at a temperature lower than the above-mentioned sol-gel transition temperature (for example, under ice cooling) to prepare a 10 wt % aqueous solution, and the aqueous solution is injected in a plastic petri dish having a diameter of 35 mm and heated to 37° C., to obtain the formation of a gel having a thickness of about 1.5 mm in the petri dish; and then the weight (f gram) of the entire petri dish containing the gel is measured. Then, the entire petri dish containing the gel was allowed to stand in 250 ml (milliliter) of water at 37° C. for 10 hours, and the weight (g gram) of the entire petri dish containing the gel was measured to evaluate the presence or absence of dissolution of the gel from the gel surface. At this time, in the hydrogel-forming polymer of the present invention, the weight loss rate of the above-mentioned gel, that is, (f-g)/f is preferably 5.0% or less, and furthermore preferably 1.0% or less (in particular 0.1% or less).

Even if the TGP aqueous solution of the present invention is gelled at a temperature higher than the above-mentioned sol-gel transition temperature, and then immersed in a large amount of water (about 0.1 to 100 times the gel in volume), the gel does not dissolve for a long period of time. Such properties of the polymer used in the present invention are achieved, for example, by the presence of two or more (a plurality of) blocks having a cloud point in the polymer.

In contrast to this, when a similar gel was prepared using the above-mentioned Pluronic (registered trademark) F-127 in which polyethylene oxide was bound to both terminals of polypropylene oxide, the present inventors have found that the gel completely dissolved in water after standing for several hours.

From the viewpoint of suppressing the cytotoxicity during non-gelation to the lowest possible level, it is preferable to use a TGP that can be gelled at a concentration with respect to water, that is, (polymer)/(polymer+water)×100(%), of 20% or less (furthermore 15% or less, in particular 10% or less).

The molecular weight of the TGP used in the present invention is preferably 30,000 or more and 30 million or less, more preferably 100,000 or more and 10 million or less, and furthermore preferably 500,000 or more and 5 million or less.

In the method of the present invention, the incubation step may be accomplished by embedding cell population in TGP solution of sol state and incubating the TGP solution in gel state at temperature higher than the sol-gel transition temperature. The incubation step may be carried out in vivo or in vitro. When incubated in vivo, TGP solution can be incubated in gel state at temperature higher than the sol-gel transition temperature by applying it to the subject in sol state. When incubated in vitro, TGP solution can be incubated by adding medium such as culture solution after gelated, in incubator, at temperature higher than the sol-gel transition temperature. In case of adding medium to TGP solution in gel state, it is possible to stably supply nutrients to the cell population in TGP solution in gel state by periodically replacing only such medium.

Incubation in vitro can be carried out under conditions normally used. For example, typical incubation conditions are 37° C. and 5% CO₂. Other temperature conditions during incubation are 25° C. to 42° C., preferably 30° C. to 40° C., more preferably 33° C. to 39° C., and especially preferably 35° C. to 38° C. Incubation period is not limited as long as cell population is not dead, and may be 1 day or longer, 4 days or longer, 8 days or longer, 16 days or longer, 21 days or longer, 24 days or longer, 32 days or longer, 48 days or longer, 64 days or longer, 72 days or longer, 90 days or longer, 102 days or longer, 114 days or longer, 126 days or longer, 140 days or longer, 160 days or longer, 180 days or longer, 250 days or longer, preferably 4 days or longer from the viewpoint of sufficient removal of senescent cell in the cell population, preferably 21 days or longer from the viewpoint of sufficient growth of stem cells, and preferably 24 days or longer from the viewpoint of sufficient tissue formation.

Incubation can be carried out in containers of any size and shape. The medium for dissolving TGP and the medium for adding to gelled TGP (sometimes referred to herein as “liquid culture medium”) are not particularly limited as long as they can maintain survival of cells, and those containing amino acids, vitamins, and electrolytes as main component can be used.

In the present invention, the medium for dissolving TGP and the medium for adding to gelled TGP may be common or different.

In one embodiment of the present invention, the medium for dissolving TGP and the medium for adding to gelled TGP may be based on a basal culture medium for cell culture. Such basal culture medium includes, but not limited to, for example, DMEM, MEM, F12, DME, RPMI 1640, MCDB (MCDB102, 104, 107, 120, 131, 153, 199, etc.), L15, SkBM, RITC80-7, CnT-PR, etc. Many of these basal culture media are commercially available and their compositions are also known. The basal culture medium may be used as it is with a standard composition (for example, as it is on the market), or the composition may be appropriately changed depending on the cell type and cell conditions. Therefore, the basal culture medium used in the present invention is not limited to those having a known composition, and it includes those in which one or more components are added, removed, increased or decreased in their amounts.

In addition to the above, the medium may contain one or more additives such as sera, growth factors (for example, FGF-2, TGF-b1, etc.), steroid preparation components, selenium components and the like.

The serum may be heterologous serum or allogeneic serum, but allogeneic serum is preferred, and autologous serum is particularly preferred among them. In addition, medium preferably does not contain therein growth factors other than those contained in the autologous serum.

The concentration of a serum is not particularly limited and the serum may be contained in a medium added to the TGP at 3%, 5%, 10% or 20%. It is preferably 10%.

In one embodiment of the present invention, the medium for dissolving TGP and the medium for adding to gelled TGP may consist of common components. Medium can contain a variety of additional components, for example, such as pharmaceutically acceptable carrier, further active component such as other senescent cell remover. Any known ones can be used as such additional components, and those skilled in the art are familiar with these additional components.

In one embodiment, the present invention further comprises cell population produced by the method of the present invention. In one embodiment, the present invention further comprises a method for treating disease in a subject, comprising applying an effective amount of cell population obtained by the present invention to subject in need thereof. Disease is not limited as long as it is the one having abnormality in living tissue.

In one embodiment, when obtained cell population is derived from cartilage tissue, the disease includes osteoarthritis, rheumatoid arthritis, osteosarcoma, whirlbone necrosis, acetabular dysplasia, meniscus injury, traumatic arthritis, and physical loss or damage of cartilage due to sports or accidents, etc.

Method for application to the subject may be transplantation of cell population.

As the transplantation, subcutaneous injection, intralymphatic injection, intravenous injection, intraarterial injection, intraperitoneal injection, intrapleural injection or direct injection into local, or direct transplantation, etc. can be listed.

In treating method of the present invention, a variety of additional components, for example, pharmaceutically acceptable carrier, component which increases take rate and further active component, etc., can be contained and applied. Any known ones can be used as such additional components, and those skilled in the art are familiar with these additional components.

The present invention is described in more detail below with specific embodiments, but is not limited thereto. In addition, part number and manufacturer of reagent were described only where they first appeared for brevity.

### EXAMPLES

### Production Example 1

The thermoreversible polymer (TGP) was obtained by following method:

42.0 g of N-isopropylacrylamide and 4.0 g of n-butyl methacrylate were dissolved in 592 g of ethanol. An aqueous solution prepared by dissolving 11.5 g of polyethylene glycol dimethacrylate (PDE6000, manufactured by NOF Corporation) in 65.1 g of water was added thereto, and the mixture was heated to 70° C. under a nitrogen stream. While maintaining 70° C. under a nitrogen stream, 0.4 mL of N,N,N′,N′-tetramethylethylenediamine (TEMED) and 4 mL of a 10% ammonium persulfate (APS) aqueous solution were added and stirred for 30 minutes. Furthermore, 0.4 mL of TEMED and 4 mL of 10% APS aqueous solution were added 4 times at intervals of 30 minutes to complete the polymerization reaction. The reaction solution was cooled to 5° C. or lower, diluted with 5 L of cold distilled water at 5° C., and concentrated to 2 L at 5° C. using an ultrafiltration membrane having a molecular weight cutoff of 100,000.

The concentrated solution was diluted by adding 4 L of cooled distilled water, and the above concentration by ultrafiltration was carried out again. The above dilution and concentration by ultrafiltration were repeated 5 times to remove those having a molecular weight of 100,000 or less.

Those not filtered by this ultrafiltration (remaining in the ultrafiltration membrane) were collected, freeze-dried, and 40 g of TGP of the present invention having a molecular weight of 100,000 or more was obtained.

### Production Example 2

1 g of TGP obtained as described above was dissolved in 9 g of distilled water under ice cooling to obtain a 10 wt % aqueous solution.

The storage elastic modulus of this aqueous solution was measured at an applied frequency of 1 Hz using a stress control type rheometer (AR500, manufactured by TA Instruments), and it was 43 Pa at 10° C., 680 Pa at 25° C., and 1310 Pa at 37° C. This temperature-dependent change in the storage elastic modulus was repeatedly and reversibly observed.

Unless otherwise stated below, the culture using TGP prepared in Production Example 1 is referred to as “3D culture”.

### Example 1

Senescent Assay in Cell Population

A portion of cartilage tissue at non-weight bearing part from a human knee joint was collected and immersed in PBS containing antibiotics (gentamicin (50 μg/ml), amphotericin (0.25 μg/ml), penicillin (100 Units/ml)/streptomycin (100 μg/ml)) for 30 minutes.

Each tissue piece was morcellated into 1 mm² or smaller with scalpel. They were digested sequentially with Tripsin-EDTA liquid and Collagenase II liquid. Cells were washed with DMEM and then, filtered and centrifuged. Cell population thus obtained was counted on a cell counting board. The number of cells was 2.1×10⁵. The cell population was 2D cultured with 10% FBS-containing DMEM in 25 cm² flask. It was 2D cultured for 14 days changing culture solution every week.

This cell population was divided into two groups, one of which was 2D cultured with above-mentioned method, and the other was embedded in the TGP prepared in Production Example 1, and 3D cultured. Both were cultured for 25 days after collection (11 days after dividing into two groups). In addition, embedding of the cell populations in TGP and 3D culture were carried out as per following method. TGP solution of low-temperature sol state was prepared by dissolving 0.5 g of TGP prepared in Production Example 1 in 5 ml of 10% FBS-containing DMEM under ice cooling condition, and the cell population was dispersed in said solution, gelated at 37° C. and embedded. Gelated TGP solution was overlain with 5 ml of aforementioned DMEM and was cultured until day 25 from collection.

The cell populations 2D cultured for 14 days (2D/Day14) and 25 days (2D/Day25), and the cell population 3D cultured for 11 days with TGP gel after 2D culture for 14 days (3D/Day25) were collected and senescence of cell was measured by measuring 8-galactosidase (SA-β-Gal) activity associated to senescence of cell. In order to apply Cell Senescence Detection Kit SPiDER-βGal (manufactured by Dojindo Laboratories) to grown cells, each cell population was treated with collagenase, made into single cells, after that each cell population was cultured in 10% FBS-containing DMEM for 1 day in 35 mm tissue culture dish, and stained. Stained cell population was measured by FACSVia (BD Biosciences) and evaluated by FLowJo analyzing software (BD Biosciences). Specifically, FSC-A vs FSC-H plots and SSC-A vs SSC-H plots were created and then, single cells were separated, fractionated by FSC value (FSC-High and FSC-Low) and fluorescence intensity was measured.

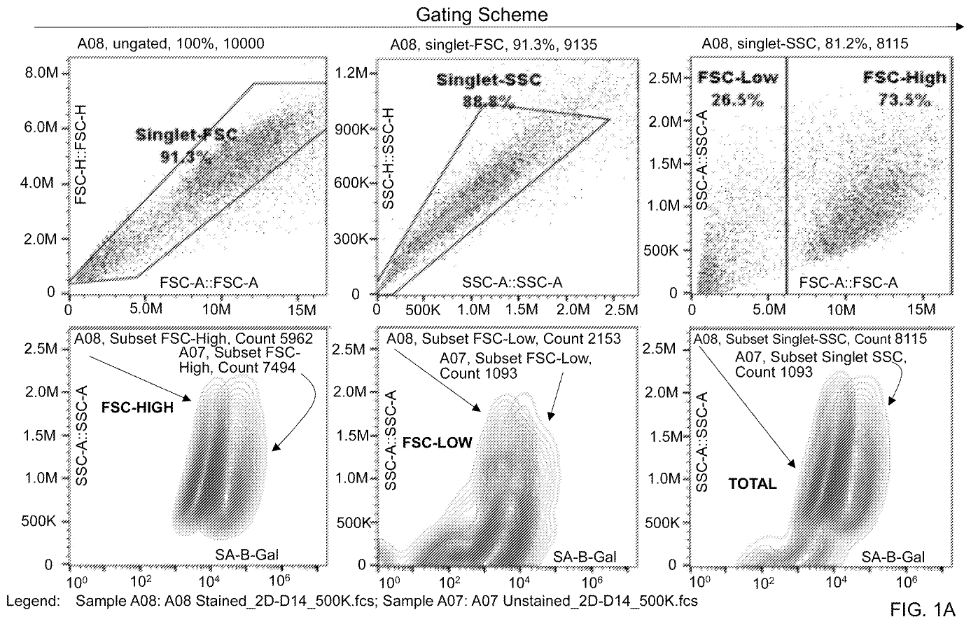

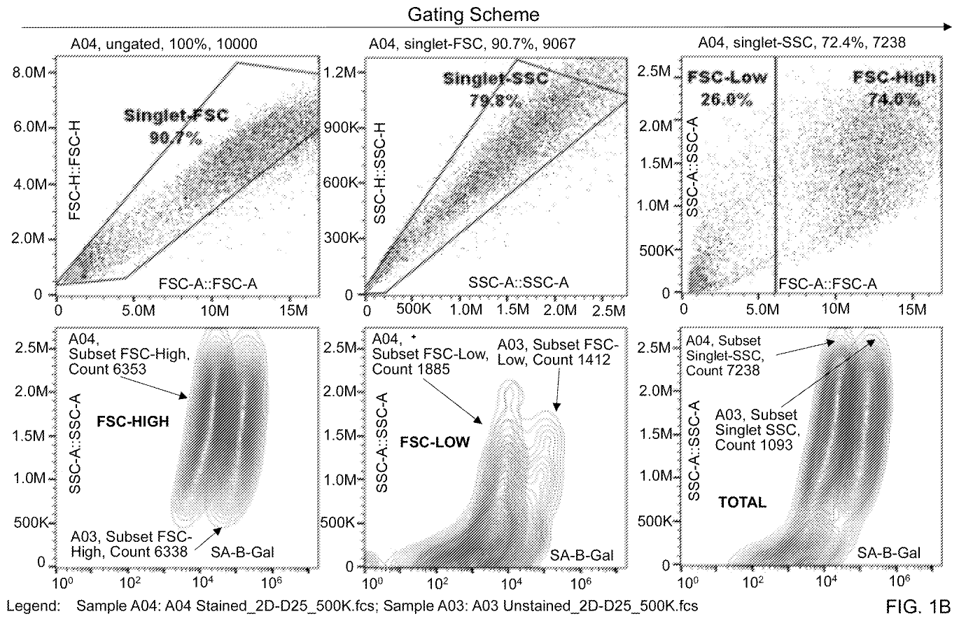

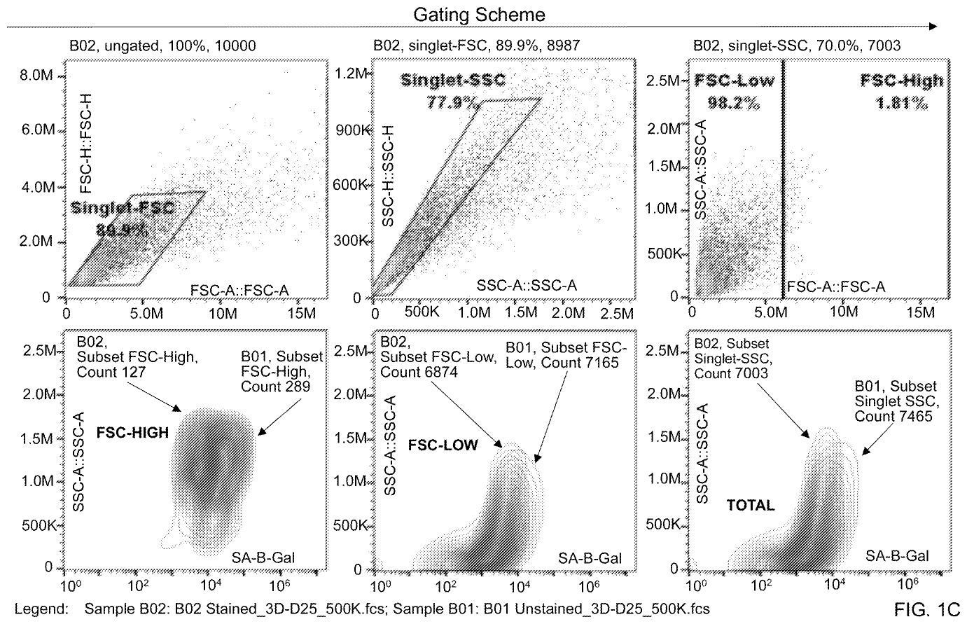

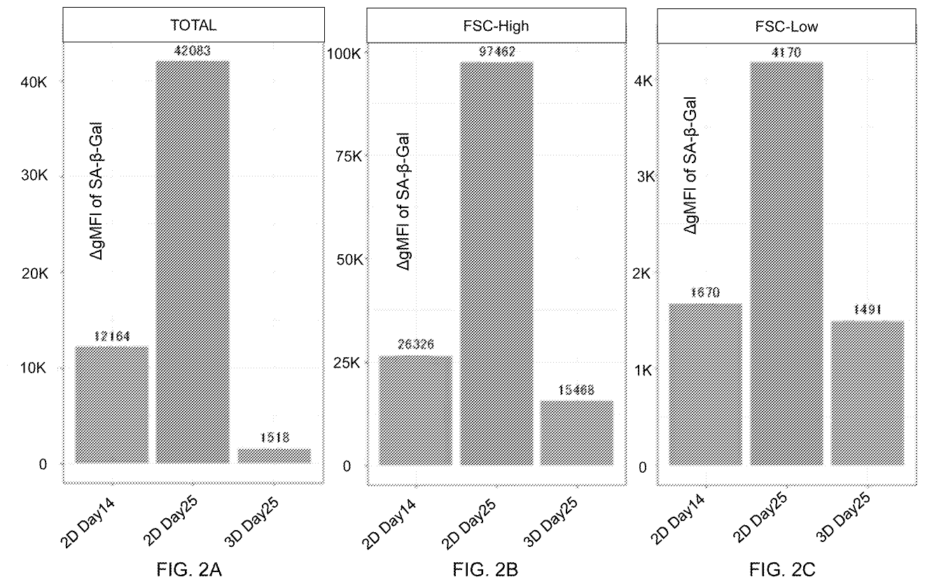

Dot plotted flow cytometry results are shown in FIG. 1. In addition, SA-β-Gal intensity of all single cells based on flow cytometry results (Total) and the SA-β-Gal intensity of all single cells fractionated by FSC-High and FSC-Low are shown in FIG. 2. It has been evident that, when cell population is 2D cultured to day 25 (2D Day25), the SA-β-Gal intensity gets higher compared to the cell population 2D cultured to day 14 (2D Day14), whereas SA-β-Gal intensity of all single cells gets lower when 3D cultured with TGP to day 25 (3D Day25). In addition, it is known that more terminally differentiated cells are contained with FSC-High and more stem cells are contained with FSC-Low, whereas 3D culture with both of FSC-High and FSC-Low resulted in lower intensity compared to cell population which was 2D cultured for 14 days (2D Day14), and so it was found that terminally differentiated cells and senescent cells in stem cells could be removed by 3D culture with TGP.

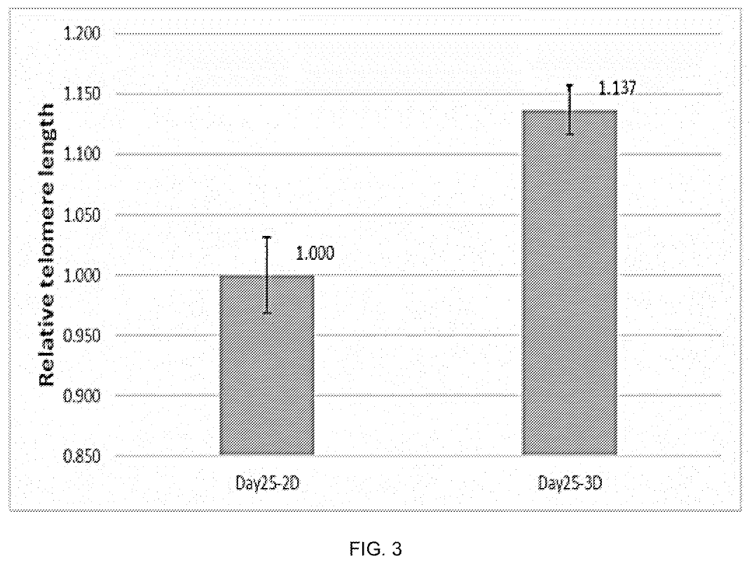

In addition, telomere length was also measured in cell populations collected on day 25 of 2D and 3D culture. The results are shown in FIG. 3. Compared to 2D culture, the average telomere length of cell population of 3D culture was longer, it is understood that senescent cells having short telomere length are removed by 3D culture.

### Example 2

Structure and Function Analysis of the Cell Population

Cell populations obtained from cartilage tissue samples derived from the subjects listed in Table 1 were obtained by the same method as Example 1, and 2D cultured and 3D cultured for 20 weeks by the same method as Example 1.

TABLE 1

| Sample Number | Age | Sex |
|---|---|---|
| 1059 | 68 | Male |
| 1064 | 79 | Female |
| 1066 | 80 | Female |
| 1067 | 66 | Female |
| 1068 | 84 | Female |
| 1069 | 73 | Female |

(1) Phase Contrast Microscope Observation

In 2D culture, spherical normal cartilage cells were identified at the beginning of the culture, but elongated fibroblast-like cells gradually began to appear as the culture progressed, and by around day 12 to day 20, they gradually began to degenerate or die, and were completely degenerated or dead by about 4 weeks after the start of culture. For example, sample 1059 died on day 28, 1066 on day 23, and 1068 on day 18.

In 3D culture, cartilage tissue-like cell populations with size of 200 to 300 μm were identified on days 3 to 5 in culture, and grew up to 500 to 1000 μm in diameter after 21 days, and growth was maintained for 20 weeks of culture period on all samples.

(2) HE Stain

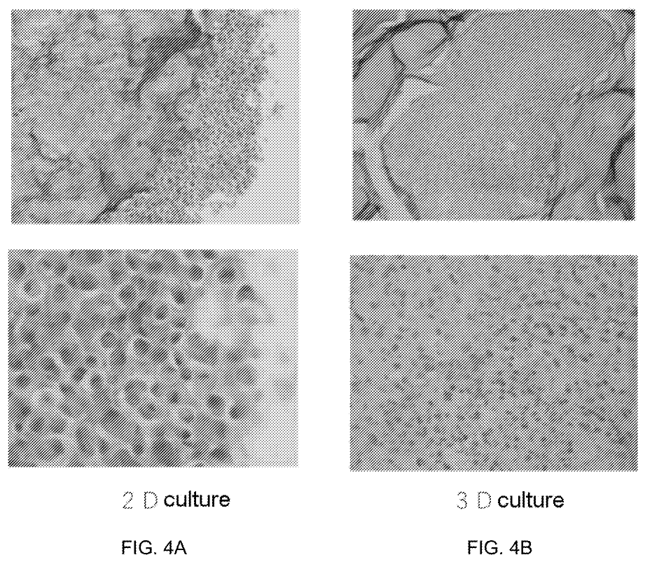

A portion of 2D cultured cell population was collected on day 25, and a portion of 3D-cultured cell population was collected on day 49 of culture, fixed in formalin, and embedded with paraffin to make block of tissue, which was then cut into 10 μm to make tissue sections. Their images observed by optical microscope (×200) are shown in FIG. 4.

ECM was not identified in 2D cultured cell population. On the other hand, it was found that 3D cultured cell population had tissue structure similar to that of healthy vitreous cartilage tissue with cartilage cells surrounded by ECM, as is typical of healthy cartilage tissue.

(3) CD44 Immunostaining

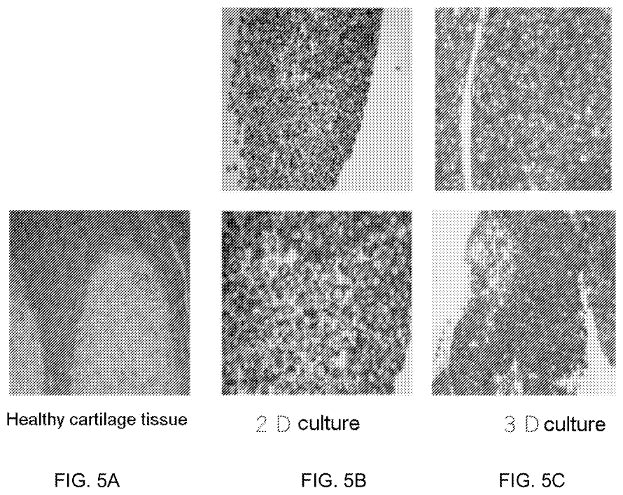

Tissue sections obtained from healthy cartilage tissue, 2D cultured cell population, and 3D cultured cell population were fixed in formalin, and embedded with paraffin to make blocks of tissue, which was then cut into 5 μm thick to make tissue sections. After deparaffinization, reaction was carried out with primary antibody rabbit monoclonal anti-CD44 antibody SP37 (Ventana Medical Systems Inc., USA) for 32 minutes at room temperature. Next, color was developed with iView DAB detection kit (Ventana Medical Systems Inc., USA). The results are shown in FIG. 5.

In 3D cultured cell population, CD44-positive cartilage calls and ECM were scattered. In 2D cultured cell population, ECM was not present and only CD44-positive cells were present.

From the above, it has been obvious that culture for long period stopped growth and healthy tissue cannot be formed in 2D cultured cell population, whereas, in 3D cultured cell population with TGP, replication capacity and differentiating capacity were maintained and healthy cartilage tissue could be formed even in cell population derived from cartilage tissue of aged people.

(4) Measurement of Gene Expression in Each Cell Population

A portion of the 2D cultured cell population was collected on days 14, 17, and 26 from start of culture. In addition, 3D cultured cell population was collected on day 42 from start of culture.

Collected cell population was added into RNAlater (RNALATER registered trademark) Stabilization Solution (Invitrogen, Cat No. AM7020) and stored at -20° C. A portion of these cell populations was purified to small RNA (RNA of 200 bases or less) with NucleoSpin (NUCLEOSPIN registered trademark) miRNA kit (TaKaRa, U0971), and then qPCRs of U6, miR-21-3p, miR-21-5p, miR-140-3p and miR-140-5p were carried out, using primers of SEQ ID NOs: 9 to 12 and mRQ 3′Primer, and U6 Forward Primer and U6 Reverse Primer (Mir-X (trademark) miRNA qRT-PCR TB Green Kit (TaKaRa Z8314N), by Thermal Cycler Dice (registered trademark) Real Time System Lite (TP700, TaKaRa). Quantitative values of each miRNA were normalized setting expression amount of U6 as 1.

In the same way, total RNA was purified using miRNAeasy Mini Kit (QIAGEN, 217004). cDNA was synthesized by reverse transcription with Superscript III reverse transcriptase (Invitrogen, 18080044) and qPCRs of GAPDH, SOX9, and COL2A1 were carried out, with primers of SEQ ID NOs: 13 to 16 and TB Green Premix Ex Taq II (Takara, Cat No. RR820S/A/B), by Thermal Cycler Dice (registered trademark) Real Time System Lite. Quantitative values of each gene were normalized setting expression amount of GAPDH as 1. Primers used are shown in Table 2 and results are shown in Tables 3 to 5.

TABLE 2

| | | SEQ |
|---|---|---|
| | | ID |
| Primer | Sequence (5′→3′) | NO |
| miR21-3p Fwd | 5′-CAACACCAGTCGATGGCTGT-3′ | 9 |
| miR21-5p Fwd | 5′-TAGCTTATCAGACTGATGTTGA-3′ | 10 |
| miR140-3p Fwd | 5′-CAGTGGTTTTACCCTATGGTAG-3′ | 11 |
| miR140-5p Fwd | 5′-TACCACAGGGTAGAACCACGG-3′ | 12 |
| Sox9 Fwd | 5′-GGAGATGAAATCTGTTCTGGGAATG-3′ | 13 |
| Sox9 Rev | 5′-ACACCAGGTTCACCAGGTTCA-3′ | 14 |
| Col2a1 Fwd | 5′-CCAGTTGGGAGTAATGCAAGGA-3′ | 15 |
| Col2a1 Rev | 5′-ACACCAGGTTCACCAGGTTCA-3′ | 16 |

TABLE 3

| Sample: 1059 | 2D-Day 126 | 3D TGP-Day 42 | fold |
|---|---|---|---|
| U6 | 1 | 1 | 1 |
| miR21-5p | 1.0968 | 1.909 | 1.74 |
| miR21-3p | 0.0273 | 0.0215 | 0.79 |
| miR140-5p | 0.0131 | 0.0656 | 5 |
| miR140-3p | 0.0281 | 0.1288 | 4.57 |
| GAPDH | 1 | 1 | 1 |
| SOX9 | 0.0041 | 0.1182 | 28.83 |

TABLE 4

| Sample: 1066 | 2D-Day 17 | 3D TGP-Day 42 | fold |
|---|---|---|---|
| U6 | 1 | 1 | 1 |
| miRNA21-5p | 0.0728 | 0.3763 | 5.17 |
| miRNA21-3p | 0.0088 | 0.0134 | 1.51 |
| miRNA140-5p | 0.0007 | 0.0248 | 31.34 |
| miRNA140-3p | 0.0024 | 0.0936 | 38.68 |

TABLE 5

| Sample: 1068 | 2D-Day 14 | 3D TGP-Day 42 | fold |
|---|---|---|---|
| U6 | 1 | 1 | 1 |
| miR21-5p | 0.816 | 1.4983 | 1.84 |
| miR21-3p | 0.037 | 0.0153 | 0.41 |
| MiR140-5p | 0.0073 | 0.0471 | 6.39 |
| miRNA140-3p | 0.0024 | 0.0936 | 38.68 |
| GAPDH | 1 | 1 | 1 |
| SOX9 | 0.0042 | 0.0791 | 18.81 |
| COL2a1 | 0.0000185 | 0.0000654 | 3.54 |

In any cell populations of 1059, 1066 and 1068, it was found that 3D cultured cell populations, compared to 2D cultured cell populations, showed increase in expression amount of miR-21 and miR-140, and significant increase in miR21-5p, miR-140-3p and miR-140-5p.

In addition, in any cell populations of 1059 and 1068 in which expression amount of SOX9 was measured, it was found that 3D cultured cell populations, compared to 2D cultured cell populations, showed significant increase in expression amount of SOX9. In addition, also in 1068 in which COL2A1 was measured, it was found that expression amount increased by 3D culture compared to 2D cultured cell population. Since miR21, miR-140, SOX9, and COL2A1 are all known that their expression increase at formation of healthy cartilage tissue, it was found that cartilage tissue having healthy function can be formed when cell population was 3D cultured.

(5) Quantification of miR140 in Liquid Culture Medium

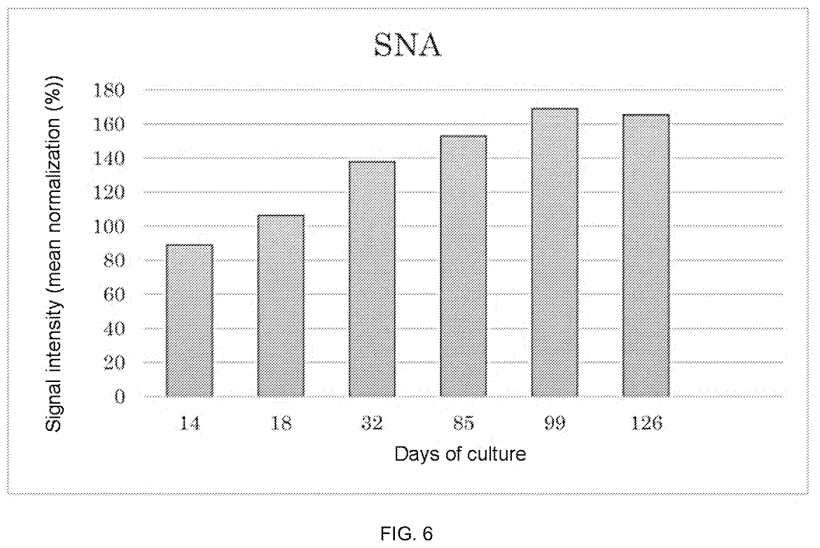

Cell populations derived from samples 1067 and 1068 were 2D cultured and 3D cultured, and 2.0 m of liquid culture medium was respectively collected on day 7 or 8 from the start of culture and stored in RNAlater (trademark) Stabilization Solution (Invitrogen, Cat No. AM7020) at −20° C. A portion of these samples were purified to small RNA (RNA of 200 bases or less) with NucleoSpin (registered trademark) miRNA kit (TaKaRa, U0971), and then qPCRs of miR-140-3p was carried out, using primers of SEQ ID NO: 3 and mRQ 3′Primer (Mir-X (trademark) miRNA qRT-PCR TB Green (registered trademark) Kit (TaKaRa Z8314N), by Thermal Cycler Dice (registered trademark) Real Time System Lite (TP700, TaKaRa). The results are shown in FIG. 6. The mean Ct value is the number of cycles before reaching at predetermined threshold, and it shows that the lower mean CT value is, the more miR-140-3p is present in the liquid culture medium. “SNP” in the table is those which were stored after centrifugation and filtration following collection, and “SNF” is those which were stored without such treatments.

TABLE 6

| | 2D | | 3D-TGP | |
|---|---|---|---|---|
| | Days of | | Days of | |
| Sample | Culture | Mean Ct Value | Culture | Mean Ct Value |
| 1067-SNP | 7 | 35.95 | 8 | 45.85 |
| 1067-SNF | 7 | 33.96 | 8 | 45.11 |
| 1068-SNP | 7 | 34.76 | 7 | 35.8 |
| 1068-SNF | 7 | 33.06 | 7 | 37.04 |

From Table 6, in both samples, it was found that miR-140-3p was more present in liquid culture medium in 3D cultured cell population compared to 2D cultured cell population. In gene expression of (4), in light of higher expression of miR-140-3p in 3D-cultured cell population compared to 2D culture, it was found that most of the miR-140-3p expressed in 3D cultured cell population was retained in cell population instead of being secreted into liquid culture medium. Thus, it has been obvious that cell population with higher miRNA content is obtained when 3D cultured.

(6) Cell Surface Glycan Analysis of Cell Constituting Cultured Cell Population

Cell population cultured in Example 1 was collected between 14 and 126 days from culture, and each TGP gel was cooled to 4° C. and collected in sol state. The samples were dispersed in Tripsin-EDTA (0.25%) solution to cells and the cells were collected. Then, they were centrifugally washed three times at 4° C. with DMEM (1800 rpm, 15 min), and each sample was cryopreserved. The sample was consigned and analyzed by Glycan Profiling Analysis Service by GlycoTechnica Ltd. Specifically, cell membrane protein was extracted from melted sample, and the membrane protein concentration was measured by BCA method (TaKaRa BCA Protein Assay Kit). Based on the measured protein concentration, PBSTx was added and diluted to protein concentration of 10 μg/mL. 100 μL of sample (10 μg/mL concentration) was added into a tube containing 100 μg of Cy3 Mono-reactive Dye Pack (GE healthcare, catalog number: PA23011), and mixed in pipette and spun down. The tube was inserted in light-shielded bag and incubated at room temperature (25° C.) for 1 hour. Then, Desalt Spin Columns (trademark) 0.5 ml (25 columns) (Thermo, catalog number: 89882) spin column was inserted in 2 mL tube and centrifuged at 1500×g for 1 minute at 4° C. to remove free Cy3. 300 μL of TBS was added to the column and centrifuged at 1500×g for 1 minute at 4° C., which process was repeated three times. The column was inserted into new 1.5 mL tube, and labeled sample (100 μL) was added to the column then centrifuged at 1500×g for 2 minutes at 4° C.

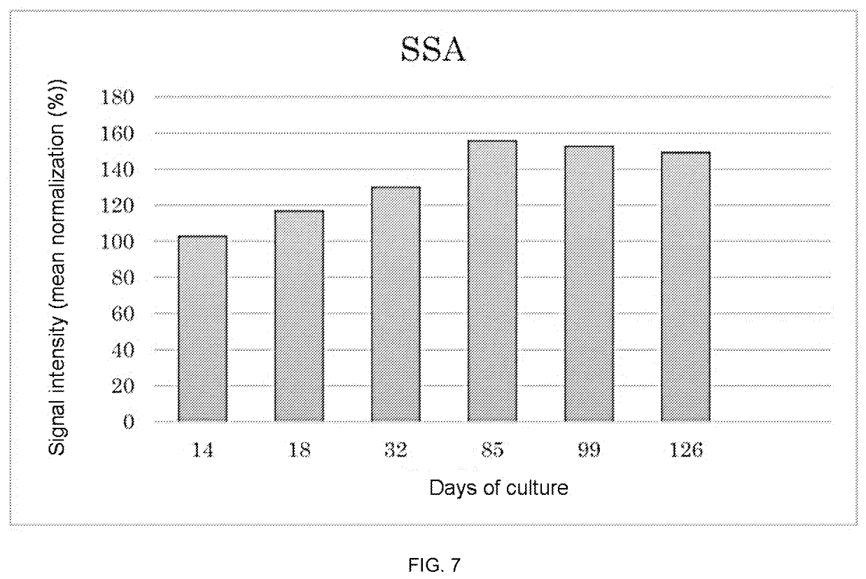

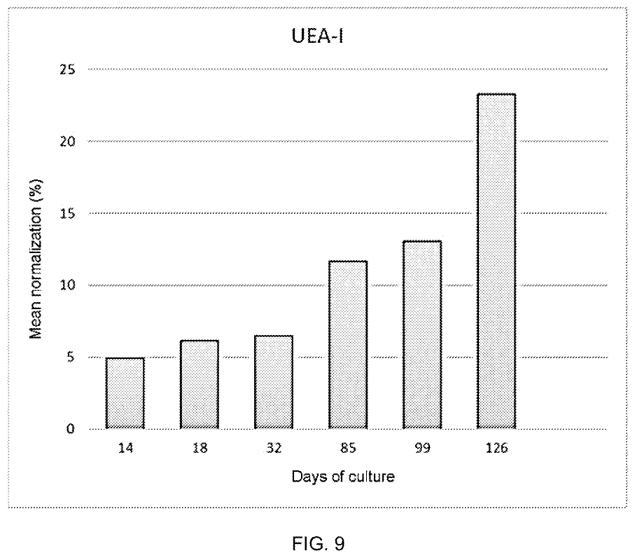

Samples were collected and 405 μL of Probing Solution (Glyco Technica) was added to obtain a sample volume of 500 μL at a concentration of 2 μg/mL. All samples were diluted to concentrations ranging from 2 μg/mL to 15.625 ng/mL using Probing Solution. Then, LecChip Ver. 1.0 (LECCHIP trademark, Glyco technica) was washed three times with Probing Solution, and the sample was added to the LecChip at 60 The sample on the LecChip was reacted at 20° C. for about 13 hours. The fluorescence pattern of the LecChip (trademark) was measured by GlycoLite 2200 (GLYCOLITE (trademark), Glyco technica) four times cumulatively, with exposure time of 1996, 2995, 3993, 4992, 6988, and 9984 (millisecond), and camera gain at fixed value. Obtained signals of 45 lectins were measured by GlycoStaion (GLYCOSTAION registered trademark) ToolsPro Suite 1.5 and, based on A. Kuno et al., J. of Proteomics & Bioinformatics, Vol. 1, May 2008, p. 68, they were divided by mean intensity of 45 lectins and multiplied by 100 to make mean normalization. Results of signal intensities for SNA, SSA, and TJA-I, UEA-1 of cell surfaces of cells constituting cell population obtained by above method are shown in Table 7 and FIGS. 6 to 9. Numbers of 14 to 126 described at top row of the table indicate the number of days of culture of the collected samples.

TABLE 7

| | 14 | 18 | 32 | 85 | 99 | 126 |
|---|---|---|---|---|---|---|
| SNA | 89.104343 | 106.46617 | 138.052 | 153.154 | 169.141 | 165.411 |
| SSA | 103.01422 | 116.85575 | 130.017 | 155.771 | 152.932 | 149.318 |
| TJA-I | 102.86368 | 121.68225 | 150.391 | 131.595 | 127.55 | 168.479 |
| UEA-I | 4.903315304 | 6.15704387 | 6.500817 | 11.64882 | 13.03299 | 23.26971 |

(Signal intensity (mean normalization (%))

From Table 7 and FIGS. 6 to 9, it is understood that, in 3D cultured cell population, signals of α2-6 sialic acid-binding lectins such as SNA, SSA and TJA-I, and α1-2 fucose-binding lectins such as UEA-1 are enhanced depending on number of days of culture. Since α2-6 sialic acid which reacts to SNA, SSA and TJA-I is a marker of somatic stem cell with high differentiating capacity (WO2016/006712A1), and α1-2 fucose binding lectin such as UEA-1 is a marker of pluripotent stem cell (Wang et al., Cell Res. 2011 November; 21(11):1551-63. doi: 10.1038/cr.2011.148. Epub 2011 Sep. 6.), it has been evident that cell population containing a lot of somatic stem cells with high differentiating capacity and pluripotent stem cells can be obtained when cell population is 3D cultured with TGP gel.

### Example 3

Morphology Analysis of Cell Population

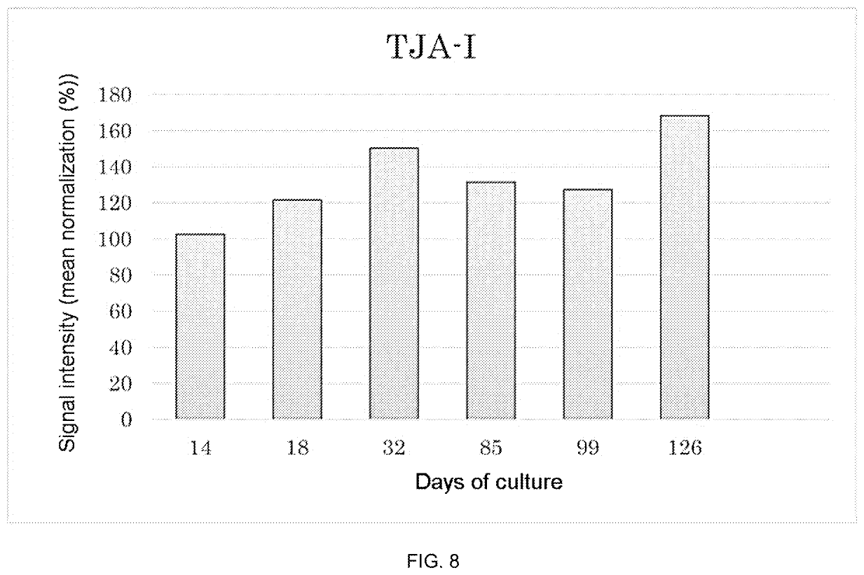

Biological tissue (buccal mucosa tissue (3 mm³)) was collected from oral cavity of healthy human. It was digested with dispase I (1000 PU/mL) at 37° C. for 60 minutes and centrifuged to collect cell population. The cell population was 2D cultured with 10% FBS-containing DMEM (2D-DMEM) and 10% FBS-containing CNTPR (2D-CNTPR) for 15 days and 3D cultured with 10% FBS-containing DMEM (3D), and morphology of cells constituting the cell population was observed. Mean values (n=10) of cell area, nuclear area, cell diameter, and nuclear diameter were analyzed using ImageJ (GitHub), and size ratios between cell and nucleus (C:N ratio) were calculated as diameter ratio and area ratio. The results are shown in FIG. 8.

TABLE 8

| | | Cell | Nuclear | Cell | Nuc | C:N | C:N | |
|---|---|---|---|---|---|---|---|---|
| | Cell | area | area | diameter | Diam | ratio | ratio | Nuc |
| | shape | (μm²) | (μm²) | (μm) | (μm) | (D ratio) | (A ratio) | Loc |
| 2D-DMEM | Elongated | 795.1 | 197.381 | 35.02 | 11.62 | 3.01 | 4.03 | NP |
| 2D-CNTPR | Spherical | 185.6 | 32.73 | 25.2 | 5.2 | 4.85 | 5.67 | NP |
| 3D | Polygonal | 3541.08 | 77.118 | 75.68 | 8.28 | 9.14 | 45.92 | NC |
| BT | Polygonal | 533 | 16.3 | 30.8 | 5.04 | 6.11 | 32.70 | NC |

Nuc. Diam. = nuclear diameter
Nuc. Loc. = nuclear localization
BT = biological tissue
NP = near the peripheral
NC = near the center
D ratio = diameter ratio
A ratio = area ratio

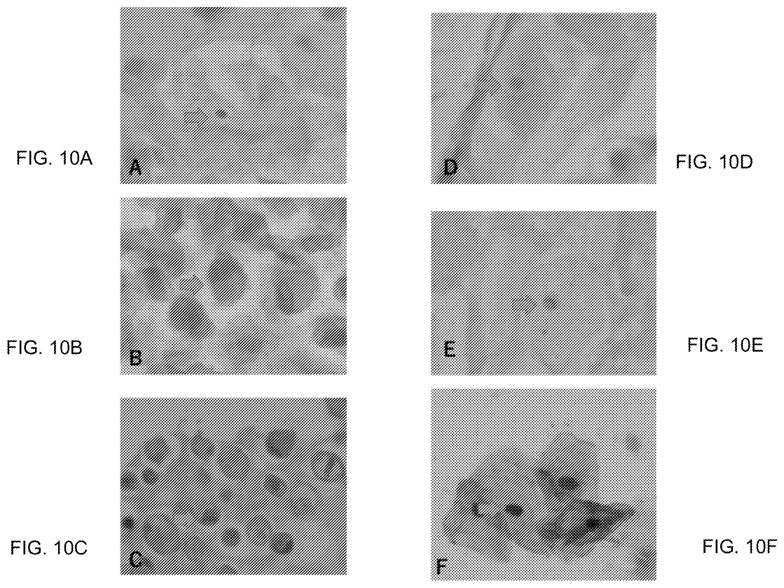

In biological tissue, cells were polygonal, nuclei were located near the center and C:N ratio (area ratio) was 32.70. In contrast, in those which were 2D cultured, cells were elongated or spherical, nuclei were located near the peripheral and C:N ratio (area ratio) was approximately 1/7 to ⅛ compared to cells in biological tissue. From these, it is understood that those which were 2D cultured have abnormal morphology compared to biological tissue. In contrast, in those which were 3D cultured, it is understood that cells are polygonal same as biological tissue, nuclei are located near the center, and C:N ratio is also high. From these, it was found that, when 3D cultured using TGP gel, cell population can maintain normal morphology similar to biological tissue. FIG. 10 shows morphologies of cell populations 2D cultured with DMEM (A and B) and CNTPR (C), cell populations 3D cultured with DMEM (D and E), and cell population immediately after separation from living organism (F). The arrows indicate positions of nuclei.

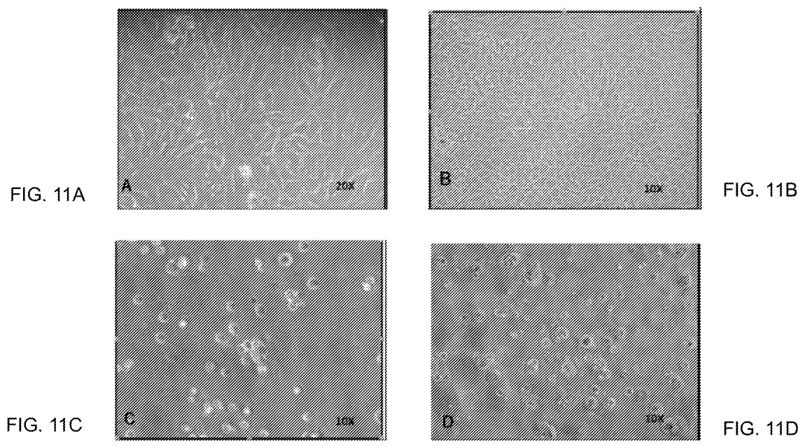

As in Example 1, cell populations derived from cartilage tissue was 2D cultured and 3D cultured for 10 days and morphological observation was carried out. In cell population immediately after isolation from organism, cells were spherical, nuclei were located near the center, and showed high C:N ratio, but in those which were 2D cultured, cells had elongated morphology, nuclei were located near the peripheral, and C:N ratio was very low compared to the cell population derived from cartilage tissue before culture. In contrast, in those which were 3D cultured, it was found that cells were spherical same as biological tissue, nuclei were located near the center, and C:N ratio was also high. From these, it was found that, when 3D cultured with TGP gel, cell population can maintain normal morphology similar to the biological tissue. FIG. 11 shows photographs of cell populations derived from cartilage tissue, 2D cultured after 7 days and after 10 days (A and B), and cell populations derived from cartilage tissue, 3D cultured after 7 days and after 10 days (C and D).

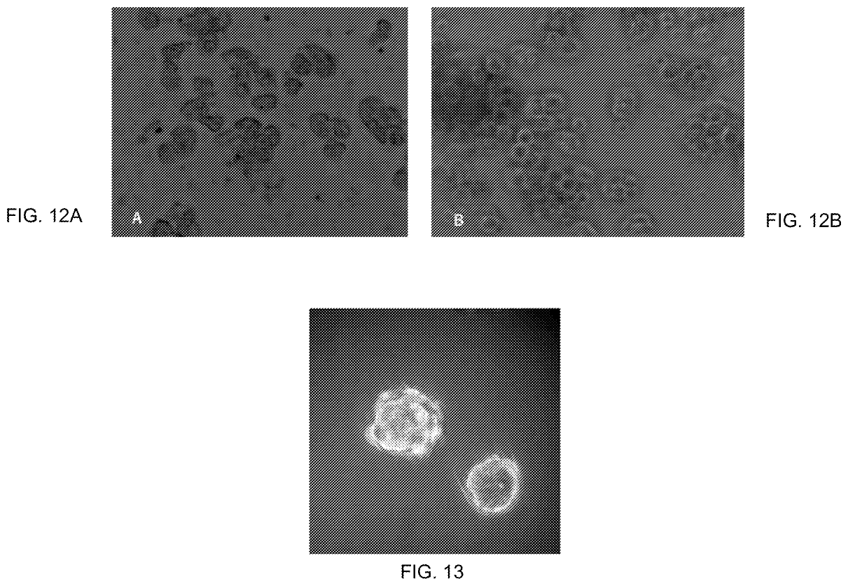

In cell population derived from liver tissue, 3D culture with MATRIGEL and TGP gel were carried out for 10 days and morphological observations was carried out. Then, in cell population derived from liver tissue before culture, cells were smooth and spherical, nuclei were located near the center and C:N ratio was high, but those which were 3D cultured with MATRIGEL had shrunken and non-smooth cell morphology, and C:N ratio was also low compared to cell population derived from liver tissue before culture. In contrast, it was found that, when 3D cultured with TGP gel, the cell population could maintain normal morphology similar to the biological tissue. FIG. 12 shows photographs, 7 days after culture, of cell population derived from liver tissue, 3D cultured with MATRIGEL (A) and cell population derived from liver tissue, 3D cultured with TGP gel (B).

When HELA cells were 2D and 3D cultured, they were elongated in 2D, but those 3D cultured with TGP gel were smooth and spherical, and C:N ratio was also high compared to 2D culture. FIG. 13 shows HELA cells 3D cultured with TGP gel.

A person skilled in the art understands that a number of various modifications can be made without deviating from the spirit of the invention. Therefore, it should be understood that the forms of the invention described herein are illustrative only and are not intended to limit the scope of the invention.

### Example 4

In cell populations 2D cultured or 3D cultured for 28 days (C8R1181), qRT-PCR was carried out to confirm expression of p16 and p21 which are known as senescence markers. C8R1181 is a cell derived from human cartilage tissue.

Total RNA was isolated from the cell population and RT-PCR was carried out with One Step TB Green PrimeScript PLUS RT-PCR kit (perfect Real Time; Takara, Japan) and Thermal Cycler Dice (registered trademark) Real Time System Lite (TP700, TaKara).

The primers used were as follows.

Primer for p16;
P16 Fwd:
(SEQ ID NO: 17)
GGCACCAGAGGCAGTAACCA
P16 Rev:
(SEQ ID NO: 18)
CCTACGCATGCCTGCTTCTACA
Primer for p21;
P21 Fwd:
(SEQ ID NO: 19)
GCGATGGAACTTCGACTTTGT
P21 Rev:
(SEQ ID NO: 20)
GGGCTTCCTCTTGGAGAAGAT

The Delta-Delta Ct method was used for mRNA quantification. The Delta-Delta Ct method is an approximation to measure relative level of mRNA between two samples by comparing with second RNA which functions as normalization standard. GADPH was used as normalization standard.

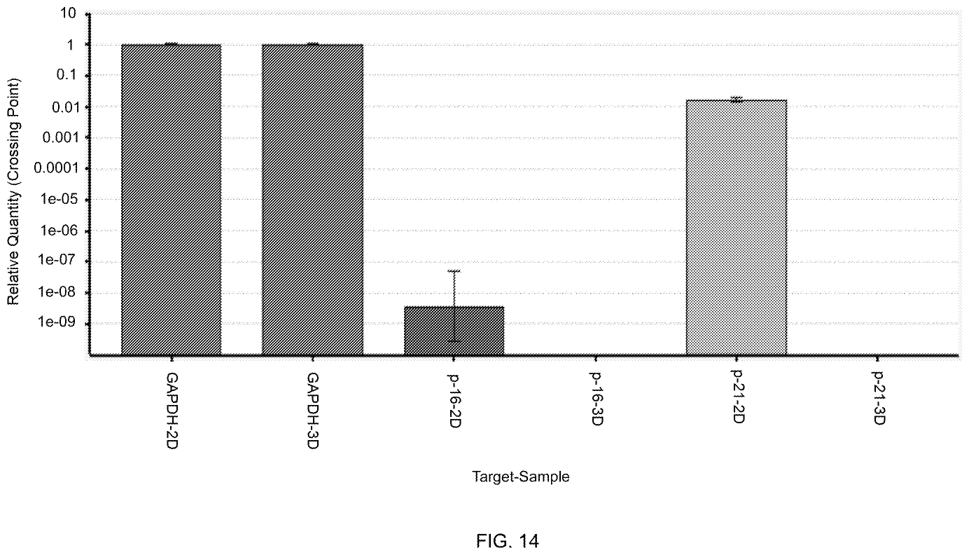

The results are shown in Table 9 and FIG. 14.

TABLE 9

C8R1181-(Relative value)

| | 2D | 3D | fold |
|---|---|---|---|
| GAPDH | 1.0000000000 | 1.0000000000 | 1.0000000000 |
| p-16 | 0.0000000037 | — | — |
| p-21 | 0.0166692400 | — | — |

In 3D cultured cell population, no PCR products were detected for either marker p16 or p21. In 2D cultured cell population, marker p16 was detected, but the amount was extremely small (detection after 40 cycles of PCR).

1-10. (canceled)

11. A method for increasing tissue regeneration capacity of a cell population, comprising a step of incubating the cell population in a thermoreversible polymer solution in a gel state.

12. The method according to claim 1, wherein the method for increasing the tissue regeneration capacity of the cell population is one or more methods selected from the group consisting of a method for removing senescent cells in the cell population, a method for maintaining the morphology of the cells constituting the cell population, a method for maintaining or increasing content of stem cells and/or somatic stem cells with high differentiating capacity in the cell population, and a method for maintaining or increasing expression of one or more genes in the biological tissue from which the cell population is derived.

13. The method according to claim 2, wherein removing senescent cells is reducing β-galactosidase positive cells in the cell population and/or extending the average telomere length of the cell population.

14. The method according to claim 2, wherein the morphology of the cells is expressed by one or more indicators selected from the group consisting of a shape of the cells constituting the cell population, nuclear localization, and size ratio between the cell and the nucleus.

15. The method according to claim 2, wherein the content of the stem cell in the cell population is α 1-2 fucose amount of the cells constituting the cell population, and the content of somatic stem cells with high differentiating capacity is α 2-6 sialic acid amount of the cells constituting the cell population.

16. The method according to claim 1, wherein one or more genes expressed in the biological tissue from which the cell population is derived are selected from SOX9, COL2A1, miR140, and miR21.

17. The method according to claim 1, wherein the cell population is a cell population derived from cartilage tissue.

18. The method according to claim 1, wherein the thermoreversible polymer is obtained by binding hydrophilic blocks and a plurality of blocks having cloud point selected from a group of a copolymer of polypropylene oxide or propylene oxide with other alkylene oxide, a copolymer of poly N-substituted acrylamide derivatives, poly N-substituted methacrylamide derivatives, N-substituted acrylamide derivatives or N-substituted methacrylamide derivatives, polyvinyl methyl ether, and partially acetylated polyvinyl alcohol.

19. A production method of cell population, comprising the method of claim 1.

20. A cell population produced by the method according to claim 9.

21. The method according to claim 1, wherein the cell population is a human cell population.

22. A method for treating disease in a subject, comprising applying an effective amount of cell population obtained by using the method according to claim 1 to the subject in need thereof.


SEQUENCE LISTING (20 sequences)

SEQ ID NO: 1 - DNA, 3931 bp, Homo sapiens
ggagagccgaaagcggagctcgaaactgactggaaacttcagtggcgcggagactcgccagtttcaaccccggaaacttttctttgcaggaggagaagagaaggggtgcaagcgcccccacttttgctctttttcctcccctcctcctcctctccaattcgcctccccccacttggagcgggcagctgtgaactggccaccccgcgccttcctaagtgctcgccgcggtagccggccgacgcgccagcttccccgggagccgcttgctccgcatccgggcagccgaggggagaggagcccgcgcctcgagtccccgagccgccgcggcttctcgcctttcccggccaccagccccctgccccgggcccgcgtatgaatctcctggaccccttcatgaagatgaccgacgagcaggagaagggcctgtccggcgcccccagccccaccatgtccgaggactccgcgggctcgccctgcccgtcgggctccggctcggacaccgagaacacgcggccccaggagaacacgttccccaagggcgagcccgatctgaagaaggagagcgaggaggacaagttccccgtgtgcatccgcgaggcggtcagccaggtgctcaaaggctacgactggacgctggtgcccatgccggtgcgcgtcaacggctccagcaagaacaagccgcacgtcaagcggcccatgaacgccttcatggtgtgggcgcaggcggcgcgcaggaagctcgcggaccagtacccgcacttgcacaacgccgagctcagcaagacgctgggcaagctctggagacttctgaacgagagcgagaagcggcccttcgtggaggaggcggagcggctgcgcgtgcagcacaagaaggaccacccggattacaagtaccagccgcggcggaggaagtcggtgaagaacgggcaggcggaggcagaggaggccacggagcagacgcacatctcccccaacgccatcttcaaggcgctgcaggccgactcgccacactcctcctccggcatgagcgaggtgcactcccccggcgagcactcggggcaatcccagggcccaccgaccccacccaccacccccaaaaccgacgtgcagccgggcaaggctgacctgaagcgagaggggcgccccttgccagaggggggcagacagccccctatcgacttccgcgacgtggacatcggcgagctgagcagcgacgtcatctccaacatcgagaccttcgatgtcaacgagtttgaccagtacctgccgcccaacggccacccgggggtgccggccacgcacggccaggtcacctacacgggcagctacggcatcagcagcaccgcggccaccccggcgagcgcgggccacgtgtggatgtccaagcagcaggcgccgccgccacccccgcagcagcccccacaggccccgccggccccgcaggcgcccccgcagccgcaggcggcgcccccacagcagccggcggcacccccgcagcagccacaggcgcacacgctgaccacgctgagcagcgagccgggccagtcccagcgaacgcacatcaagacggagcagctgagccccagccactacagcgagcagcagcagcactcgccccaacagatcgcctacagccccttcaacctcccacactacagcccctcctacccgcccatcacccgctcacagtacgactacaccgaccaccagaactccagctcctactacagccacgcggcaggccagggcaccggcctctactccaccttcacctacatgaaccccgctcagcgccccatgtacacccccatcgccgacacctctggggtcccttccatcccgcagacccacagcccccagcactgggaacaacccgtctacacacagctcactcgaccttgaggaggcctcccacgaagggcgaagatggccgagatgatcctaaaaataaccgaagaaagagaggaccaaccagaattccctttggacatttgtgtttttttgtttttttattttgttttgttttttcttcttcttcttcttccttaaagacatttaagctaaaggcaactcgtacccaaatttccaagacacaaacatgacctatccaagcgcattacccacttgtggccaatcagtggccaggccaaccttggctaaatggagcagcgaaatcaacgagaaactggactttttaaaccctcttcagagcaagcgtggaggatgatggagaatcgtgtgatcagtgtgctaaatctctctgcctgtttggactttgtaattatttttttagcagtaattaaagaaaaaagtcctctgtgaggaatattctctattttaaatatttttagtatgtactgtgtatgattcattaccattttgaggggatttatacatatttttagataaaattaaatgctcttatttttccaacagctaaactactcttagttgaacagtgtgccctagcttttcttgcaaccagagtatttttgtacagatttgctttctcttacaaaaagaaaaaaaaaatcctgttgtattaacatttaaaaacagaattgtgttatgtgatcagttttgggggttaactttgcttaattcctcaggctttgcgatttaaggaggagctgccttaaaaaaaaataaaggccttattttgcaattatgggagtaaacaatagtctagagaagcatttggtaagctttatcatatatatattttttaaagaagagaaaaacaccttgagccttaaaacggtgctgctgggaaacatttgcactcttttagtgcatttcctcctgcctttgcttgttcactgcagtcttaagaaagaggtaaaaggcaagcaaaggagatgaaatctgttctgggaatgtttcagcagccaataagtgcccgagcacactgcccccggttgcctgcctgggccccatgtggaaggcagatgcctgctcgctctgtcacctgtgcctctcagaacaccagcagttaaccttcaagacattccacttgctaaaattatttattttgtaaggagaggttttaattaaaacaaaaaaaaattcttttttttttttttttccaattttaccttctttaaaataggttgttggagctttcctcaaagggtatggtcatctgttgttaaattatgttcttaactgtaaccagtttttttttatttatctctttaatctttttttattattaaaagcaagtttctttgtattcctcaccctagatttgtataaatgcctttttgtccatcccttttttctttgttgtttttgttgaaaacaaactggaaacttgtttctttttttgtataaatgagagattgcaaatgtagtgtatcactgagtcatttgcagtgttttctgccacagacctttgggctgccttatattgtgtgtgtgtgtgggtgtgtgtgtgttttgacacaaaaacaatgcaagcatgtgtcatccatatttctctgcatcttctcttggagtgagggaggctacctggaggggatcagcccactgacagaccttaatcttaattactgctgtggctagagagtttgaggattgctttttaaaaaagacagcaaacttttttttttatttaaaaaaagatatattaacagttttagaagtcagtagaataaaatcttaaagcactcataatatggcatccttcaatttctgtataaaagcagatctttttaaaaagatacttctgtaacttaagaaacctggcatttaaatcatattttgtctttaggtaaaagctttggtttgtgttcgtgttttgtttgtttcacttgtttccctcccagccccaaaccttttgttctctccgtgaaacttacctttccctttttctttctcttttttttttttgtatattattgtttacaataaatatacattgcattaaaaa

SEQ ID NO: 2 - DNA, 5059 bp, Homo sapiens
gcagagcgctgctgggctgccgggtctcccgcttccccctcctgctccaagggcctcctgcatgagggcgcggtagagacccggacccgcgccgtgctcctgccgtttcgctgcgctccgcccgggcccggctcagccaggccccgcggtgagccatgattcgcctcggggctccccagacgctggtgctgctgacgctgctcgtcgccgctgtccttcggtgtcagggccaggatgtccaggaggctggcagctgtgtgcaggatgggcagaggtataatgataaggatgtgtggaagccggagccctgccggatctgtgtctgtgacactgggactgtcctctgcgacgacataatctgtgaagacgtgaaagactgcctcagccctgagatccccttcggagagtgctgccccatctgcccaactgacctcgccactgccagtgggcaaccaggaccaaagggacagaaaggagaacctggagacatcaaggatattgtaggacccaaaggacctcctgggcctcagggacctgcaggggaacaaggacccagaggggatcgtggtgacaaaggtgaaaaaggtgcccctggacctcgtggcagagatggagaacctgggacccctggaaatcctggcccccctggtcctcccggcccccctggtccccctggtcttggtggaaactttgctgcccagatggctggaggatttgatgaaaaggctggtggcgcccagttgggagtaatgcaaggaccaatgggccccatgggacctcgaggacctccaggccctgcaggtgctcctgggcctcaaggatttcaaggcaatcctggtgaacctggtgaacctggtgtctctggtcccatgggtccccgtggtcctcctggtccccctggaaagcctggtgatgatggtgaagctggaaaacctggaaaagctggtgaaaggggtccgcctggtcctcagggtgctcgtggtttcccaggaaccccaggccttcctggtgtcaaaggtcacagaggttatccaggcctggacggtgctaagggagaggcgggtgctcctggtgtgaagggtgagagtggttccccgggtgagaacggatctccgggcccaatgggtcctcgtggcctgcctggtgaaagaggacggactggccctgctggcgctgcgggtgcccgaggcaacgatggtcagccaggccccgcagggcctccgggtcctgtcggtcctgctggtggtcctggcttccctggtgctcctggagccaagggtgaagccggccccactggtgcccgtggtcctgaaggtgctcaaggtcctcgcggtgaacctggtactcctgggtcccctgggcctgctggtgcctccggtaaccctggaacagatggaattcctggagccaaaggatctgctggtgctcctggcattgctggtgctcctggcttccctgggccacggggccctcctggccctcaaggtgcaactggtcctctgggcccgaaaggtcagacgggtgaacctggtattgctggcttcaaaggtgaacaaggccccaagggagaacctggccctgctggcccccagggagcccctggacccgctggtgaagaaggcaagagaggtgcccgtggagagcctggtggcgttgggcccatcggtccccctggagaaagaggtgctcccggcaaccgcggtttcccaggtcaagatggtctggcaggtcccaagggagcccctggagagcgagggcccagtggtcttgctggccccaagggagccaacggtgaccctggccgtcctggagaacctggccttcctggagcccggggtctcactggccgccctggtgatgctggtcctcaaggcaaagttggcccttctggagcccctggtgaagatggtcgtcctggacctccaggtcctcagggggctcgtgggcagcctggtgtcatgggtttccctggccccaaaggtgccaacggtgagcctggcaaagctggtgagaagggactgcctggtgctcctggtctgaggggtcttcctggcaaagatggtgagacaggtgctgcaggaccccctggccctgctggacctgctggtgaacgaggcgagcagggtgctcctgggccatctgggttccagggacttcctggccctcctggtcccccaggtgaaggtggaaaaccaggtgaccagggtgttcccggtgaagctggagcccctggcctcgtgggtcccaggggtgaacgaggtttcccaggtgaacgtggctctcccggtgcccagggcctccagggtccccgtggcctccccggcactcctggcactgatggtcccaaaggtgcatctggcccagcaggcccccctggggctcagggccctccaggtcttcagggaatgcctggcgagaggggagcagctggtatcgctgggcccaaaggcgacaggggtgacgttggtgagaaaggccctgagggagcccctggaaaggatggtggacgaggcctgacaggtcccattggcccccctggcccagctggtgctaatggcgagaagggagaagttggacctcctggtcctgcaggaagtgctggtgctcgtggcgctccgggtgaacgtggagagactgggccccccggaccagcgggatttgctgggcctcctggtgctgatggccagcctggggccaagggtgagcaaggagaggccggccagaaaggcgatgctggtgcccctggtcctcagggcccctctggagcacctgggcctcagggtcctactggagtgactggtcctaaaggagcccgaggtgcccaaggccccccgggagccactggattccctggagctgctggccgcgttggacccccaggctccaatggcaaccctggaccccctggtccccctggtccttctggaaaagatggtcccaaaggtgctcgaggagacagcggcccccctggccgagctggtgaacccggcctccaaggtcctgctggaccccctggcgagaagggagagcctggagatgacggtccctctggtgccgaaggtccaccaggtccccagggtctggctggtcagagaggcatcgtcggtctgcctgggcaacgtggtgagagaggattccctggcttgcctggcccgtcgggtgagcccggcaagcagggtgctcctggagcatctggagacagaggtcctcctggccccgtgggtcctcctggcctgacgggtcctgcaggtgaacctggacgagagggaagccccggtgctgatggcccccctggcagagatggcgctgctggagtcaagggtgatcgtggtgagactggtgctgtgggagctcctggagcccctgggccccctggctcccctggccccgctggtccaactggcaagcaaggagacagaggagaagctggtgcacaaggccccatgggaccctcaggaccagctggagcccggggaatccagggtcctcaaggccccagaggtgacaaaggagaggctggagagcctggcgagagaggcctgaagggacaccgtggcttcactggtctgcagggtctgcccggccctcctggtccttctggagaccaaggtgcttctggtcctgctggtccttctggccctagaggtcctcctggccccgtcggtccctctggcaaagatggtgctaatggaatccctggccccattgggcctcctggtccccgtggacgatcaggcgaaaccggccctgctggtcctcctggaaatcctggaccccctggtcctccaggtccccctggccctggcatcgacatgtccgcctttgctggcttaggcccgagagagaagggccccgaccccctgcagtacatgcgggccgaccaggcagccggtggcctgagacagcatgacgccgaggtggatgccacactcaagtccctcaacaaccagattgagagcatccgcagccccgagggctcccgcaagaaccctgctcgcacctgcagagacctgaaactctgccaccctgagtggaagagtggagactactggattgaccccaaccaaggctgcaccttggacgccatgaaggttttctgcaacatggagactggcgagacttgcgtctaccccaatccagcaaacgttcccaagaagaactggtggagcagcaagagcaaggagaagaaacacatctggtttggagaaaccatcaatggtggcttccatttcagctatggagatgacaatctggctcccaacactgccaacgtccagatgaccttcctacgcctgctgtccacggaaggctcccagaacatcacctaccactgcaagaacagcattgcctatctggacgaagcagctggcaacctcaagaaggccctgctcatccagggctccaatgacgtggagatccgggcagagggcaatagcaggttcacgtacactgccctgaaggatggctgcacgaaacataccggtaagtggggcaagactgttatcgagtaccggtcacagaagacctcacgcctccccatcattgacattgcacccatggacataggagggcccgagcaggaattcggtgtggacatagggccggtctgcttcttgtaaaaacctgaacccagaaacaacacaatccgttgcaaacccaaaggacccaagtactttccaatctcagtcactctaggactctgcactgaatggctgacctgacctgatgtccattcatcccaccctctcacagttcggacttttctcccctctctttctaagagacctgaactgggcagactgcaaaataaaatctcggtgttctatttatttattgtcttcctgtaagaccttcgggtcaaggcagaggcaggaaactaactggtgtgagtcaaatgccccctgagtgactgcccccagcccaggccagaagacctcccttcaggtgccgggcgcaggaactgtgtgtgtcctacacaatggtgctattctgtgtcaaacacctctgtattttttaaaacatcaattgatattaaaaatgaaaagattattggaaagta

SEQ ID NO: 3 - RNA, 100 nt, Homo sapiens
ugugucucucucuguguccugccagugguuuuacccuaugguagguuacgucaugcuguucuaccacaggguagaaccacggacaggauaccggggcacc

SEQ ID NO: 4 - RNA, 21 nt, Homo sapiens
uaccacaggguagaaccacgg

SEQ ID NO: 5 - RNA, 22 nt, Homo sapiens
cagugguuuuacccuaugguag

SEQ ID NO: 6 - RNA, 72 nt, Homo sapiens
ugucggguagcuuaucagacugauguugacuguugaaucucauggcaacaccagucgaugggcugucugaca

SEQ ID NO: 7 - RNA, 21 nt, Homo sapiens
caacaccagucgaugggcugu

SEQ ID NO: 8 - RNA, 22 nt, Homo sapiens
uagcuuaucagacugauguuga

SEQ ID NO: 9 - DNA, 20 bp, Artificial Sequence
  Forward primer for miR21-3p
caacaccagtcgatggctgt

SEQ ID NO: 10 - DNA, 22 bp, Artificial Sequence
  Forward primer for miR21-5p
tagcttatcagactgatgttga

SEQ ID NO: 11 - DNA, 21 bp, Artificial Sequence
  Forward primer for miR140-3p
cagtggttttaccctatggta

SEQ ID NO: 12 - DNA, 21 bp, Artificial Sequence
  Forward primer for miR140-5p
taccacagggtagaaccacgg

SEQ ID NO: 13 - DNA, 25 bp, Artificial Sequence
  Forward primer for SOX9
ggagatgaaatctgttctgggaatg

SEQ ID NO: 14 - DNA, 25 bp, Artificial Sequence
  Reverse primer for SOX9
ttgaaggttaactgctggtgttctg

SEQ ID NO: 15 - DNA, 22 bp, Artificial Sequence
  Forward primer for COL2A1
ccagttgggagtaatgcaagga

SEQ ID NO: 16 - DNA, 21 bp, Artificial Sequence
  Reverse primer for COL2A1
acaccaggttcaccaggttca

SEQ ID NO: 17 - DNA, 20 bp, Artificial Sequence
  Forward primer for p16
ggcaccagaggcagtaacca

SEQ ID NO: 18 - DNA, 22 bp, Artificial Sequence
  Reverse primer for p16
cctacgcatgcctgcttctaca

SEQ ID NO: 19 - DNA, 21 bp, Artificial Sequence
  Forward primer for p21
gcgatggaacttcgactttgt

SEQ ID NO: 20 - DNA, 21 bp, Artificial Sequence
  Reverse primer for p21
gggcttcctcttggagaagat